(19) United States
(12) Patent Application Publication
(10) Pub. No.: US 2016/0046682 A1
(43) Pub. Date: Feb. 18, 2016

(54) ARTIFICIAL TRANSCRIPTION FACTORS ENGINEERED TO OVERCOME ENDOSOMAL ENTRAPMENT

(71) Applicant: ALIOPHTHA AG, Basel (CH)

(72) Inventors: Albert NEUTZNER, Schliengen (DE); Josef FLAMMER, Binningen (CH); Alice HUXLEY, Binningen (CH)

(73) Assignee: ALIOPHTHA AG, Basel (CH)

(21) Appl. No.: 14/781,701

(22) PCT Filed: Apr. 2, 2014
(86) PCT No.: PCT/EP2014/056589

§ 371 Date: Oct. 1, 2015

(30) Foreign Application Priority Data
Apr. 3, 2013 (EP) 13162197.1

(51) Int. Cl. C07K 14/47

(52) U.S. Cl. CPC C07K 14/4702

(57) ABSTRACT

The invention relates to an artificial transcription factor comprising a polydactyl zinc finger protein targeting specifically a gene promoter, engineered to overcome endosomal entrapment after transduction into cells. Such artificial transcription factor comprises a polydactyl zinc finger protein fused to an inhibitory or activatory protein domain, a nuclear localization sequence, a protein transduction domain, and an endosome-specific protease-recognition site. These transducible artificial transcription factors are particularly useful for the treatment of diseases caused or modulated by membrane-bound receptor proteins, nuclear receptor proteins or products of haploinsufficient genes.

### FIELD OF THE INVENTION

The invention relates to an artificial transcription factor comprising a polydactyl zinc finger protein targeting specifically a gene promoter and a protein transduction domain engineered to overcome endosomal entrapment after transduction into cells.

### BACKGROUND OF THE INVENTION

Artificial transcription factors are proposed to be useful tools for modulating gene expression (Sera T., 2009, Adv Drug Deliv Rev 61, 513-526). Many naturally occurring transcription factors, influencing gene expression either through repression or activation of gene transcription, possess complex specific domains for the recognition of a certain DNA sequence. This makes them unattractive targets for manipulation if one intends to modify their specificity and target gene(s). However, a certain class of transcription factors contains several so called zinc finger (ZF) domains, which are modular and therefore lend themselves to genetic engineering. Zinc fingers are short (30 amino acids) DNA binding motifs targeting almost independently three DNA base pairs. A protein containing several such zinc fingers fused together is thus able to recognize longer DNA sequences. A hexameric zinc finger protein (ZFP) recognizes an 18 base pairs (bp) DNA target, which is almost unique in the entire human genome. Initially thought to be completely context independent, more in-depth analyses revealed some context specificity for zinc fingers (Klug A., 2010, Annu Rev Biochem 79, 213-231). Mutating certain amino acids in the zinc finger recognition surface altering the binding specificity of ZF modules resulted in defined ZF building blocks for most of 5′-GNN-3′, 5′-CNN-3′, 5′-ANN-3′, and some 5′-TNN-3′ codons (e.g. so-called Barbas modules, see Dreier B., Barbas C. F. 3^{rd} et al., 2005, J Biol Chem 280, 35588-35597). While early work on artificial transcription factors concentrated on a rational design based on combining preselected zinc fingers with a known 3 bp target sequence, the realization of a certain context specificity of zinc fingers necessitated the generation of large zinc finger libraries which are interrogated using sophisticated methods such as bacterial or yeast one hybrid, phage display, compartmentalized ribosome display or in vivo selection using FACS analysis.

Using such artificial zinc finger proteins, DNA loci within the human genome can be targeted with high specificity. Thus, these zinc finger proteins are ideal tools to transport protein domains with transcription-modulatory activity to specific promoter sequences resulting in the modulation of expression of a gene of interest. Suitable domains for the silencing of transcription are the Krueppel-associated domain (KRAB) as N-Terminal (SEQ ID NO: 1) or C-terminal (SEQ ID NO: 2) KRAB domain, the Sin3-interacting domain (SID, SEQ ID NO: 3) and the ERF repressor domain (ERD, SEQ ID NO: 4), while activation of gene transcription is achieved through herpes virus simplex VP16 (SEQ ID NO: 5) or VP64 (tetrameric repeat of VP16, SEQ ID NO: 6) domains (Beerli R. R. et al., 1998, Proc Natl Acad Sci USA 95, 14628-14633). Additional domains considered to confer transcriptional activation are CJ7 (SEQ ID NO: 7), p65-TA1 (SEQ ID NO: 8), SAD (SEQ ID NO: 9), NF-1 (SEQ ID NO: 10), AP-2 (SEQ ID NO: 11), SP1-A (SEQ ID NO: 12), SP1-B (SEQ ID NO: 13), Oct-1 (SEQ ID NO: 14), Oct-2 (SEQ ID NO: 15), Oct-2_{—}5 x (SEQ ID NO: 16), MTF-1 (SEQ ID NO: 17), BTEB-2 (SEQ ID NO: 18) and LKLF (SEQ ID NO: 19). In addition, transcriptionally active domains of proteins defined by gene ontology GO: 0001071 (http://amigo.geneontology.org/cgi-bin/amigo/term_details?term=GO:0001071) are considered to achieve transcriptional regulation of target proteins. Fusion proteins comprising engineered zinc finger proteins as well as regulatory domains are referred to as artificial transcription factors.

While small molecule drugs are not always able to selectively target a certain member of a given protein family due to the high conservation of specific features, biologicals offer great specificity as shown for antibody-based novel drugs. However, virtually all biologicals to date act extracellularly. Especially above mentioned artificial transcription factors would be suitable to influence gene transcription in a therapeutically useful way. However, the delivery of such factors to the site of action—the nucleus—is not easily achieved, thus hampering the usefulness of therapeutic artificial transcription factor approaches, e.g. by relaying on retroviral delivery with all the drawbacks of this method such as immunogenicity and the potential for cellular transformation (Lund C. V. et al., 2005, Mol Cell Biol 25, 9082-9091).

So called protein transduction domains (PTDs) were shown to promote protein translocation across the plasma membrane into the cytosol/nucleoplasm. Short peptides such as the HIV derived TAT peptide (SEQ ID NO: 20), mT02 (SEQ ID NO: 21), mTO3 (SEQ ID NO: 22), R9 (SEQ ID NO: 23), ANTP (SEQ ID NO: 24) and others were shown to induce a cell-type independent macropinocytotic uptake when fused to cargo proteins (Wadia J. S. et al., 2004, Nat Med 10, 310-315). Upon arrival in the cytosol, such fusion proteins were shown to have biological activity. Interestingly, even misfolded proteins can become functional following protein transduction most likely through the action of intracellular chaperones. However, a major hurdle for the use of protein transduction domains for delivering therapeutic cargo to cells is the limited escape of such proteins from the endosomal compartment to other subcellular localization such as the nucleus (Koren E and Torchilin V. P., 2012, Trends in Mol Med 18, 385-393). The need to increase the endosomal escape of cargo proteins following protein transduction is long recognized, and two major approaches to enhance endosomal escape are used: First, co-delivery of so called fusogenic peptides such as HA2 (SEQ ID NO: 25), KALA (SEQ ID NO: 26) or GALA (SEQ ID NO: 27) was shown to increase protein transduction into the cytosol of cells. Once inside the endosome, these peptides are capable of interacting with the endosomal membrane leading to the rupture of these vesicles liberating their contents. Second, lysosomotropic agents such as chloroquine known to disrupt the endosomal compartment were shown to increase escape of cargo proteins from endosomes. Other approaches to increase endosomal escape involve fusogenic lipids and membrane-disruptive polymers such as PEI (El-Sayed A. et al., 2009, AAPS J 11, 13-22). So far, all approaches to increase endosomal escape of cargo proteins following protein transduction involve an agent capable of disrupting the endosomal membrane.

A large percentage of all known drug targets are receptor molecules that are either stimulated or blocked by the action of small molecule drugs with oftentimes considerable off-target activities. Examples for such receptors are the histamine H1 receptor or alpha- and beta-adrenoreceptors, but in general proteins defined by gene ontology GO:0004888 and GO:0004930.

The vasoactive endothelin system plays an important role in the pathogenesis of various diseases. Endothelins, on the one hand, are involved in the regulation of blood supply and, on the other hand, are main players in the cascade of events induced by hypoxia. Endothelin is e. g. involved in the breakdown of the blood-brain or the blood-retina barrier and in the neovascularisation. Endothelin is furthermore involved in neurodegeneration but also the regulation of the threshold of pain sensation or even thirst feeling. Endothelin is also involved in regulation of intraocular pressure.

The action of endothelin is mediated by its cognate receptors, mainly endothelin receptor A, usually located on smooth muscle cells surrounding blood vessels. Influencing the endothelin system—systemically or locally—is of interest for the treatment of many diseases such as subarachnoidal or brain hemorrhages. Endothelin also influences the course of multiple sclerosis. Endothelin contributes to (pulmonary) hypertension, but also to arterial hypotension, cardiomyopathy and to Raynaud syndrome, variant angina and other cardiovascular diseases. Endothelin is involved in diabetic nephropathy and diabetic retinopathy. In the eye it further plays a role for the glaucomatous neurodegeneration, retinal vein occlusion, giant cell arthritis, retinitis pigmentosa, age related macula degeneration, central serous chorioretinopathy, Morbus Leber, Susac syndrome, intraocular hemorrhages, epiretinal gliosis and certain other pathological conditions.

The eye is an exquisite organ that strongly relies on a balanced and sufficient perfusion to meet its high oxygen demand. Failure to provide sufficient and stable oxygen supply causes ischemia-reperfusion injury leading to glial activation and neuronal damage as observed in glaucoma patients with progressing disease despite normal or normalized intraocular pressure. Insufficient blood supply also leads to hypoxia causing run-away neovascularization with the potential of further retinal damage as evident during diabetic retinopathy or wet age related macular degeneration. Eye tissue perfusion is under complex control and depends on blood pressure, intraocular pressure as well as local factors modulating vessel diameter. Such local factors are for example the mentioned endothelins, short peptides with a strong vasoconstrictive activity. Three isoforms of endothelins (ET-1, ET-2, and ET-3) are produced by endothelin converting enzyme from precursor molecules secreted by endothelial cells localized in the blood vessel wall. Two cognate receptors for mature ET are known, ETRA and ETRB. While ETRA is localized to smooth muscle cells forming vessels walls and promoting vasoconstriction, ETRB is mainly expressed on endothelial cells and acts vasodilatatory by promoting the release of nitric oxide, thus causing smooth muscle relaxation. ETRA and ETRB belong to the large class of G-protein coupled seven transmembrane helix receptors. The binding of ET to ETRA or ETRB results in G protein activation, thus triggering an increase in intracellular calcium concentration and thereby causing a wide array of cellular reactions.

Influencing the ET system pharmacologically might prove useful in cases, wherein ET levels are elevated and ETs act in a detrimental fashion, such as during retinal vein occlusion, glaucomatous neurodegeneration, retinitis pigmentosa, giant cell arteritis, central serous chorioretinopathy, multiple sclerosis, optic neuritis, rheumatoid arthritis, Susac syndrome, radiation retinopathy, epiretinal gliosis, fibromyalgia and diabetic retinopathy. To this end, down-regulation of ETRA will aid to modulate disease outcome. But under certain circumstances, upregulation of ETRA and therefore an increased sensitivity towards ET might be desirable, for example to promote corneal wound healing during the recovery from corneal trauma or corneal ulcer.

ETRB-mediated signaling is connected to pathophysiological processes e.g. during cancer stem cell maintenance and tumor growth. In addition, upregulation of ETRB is associated with glaucomatous neurodegeneration while inhibition of ETRB was shown to act neuroprotective during glaucoma. Furthermore, ETRB is upregulated during inflammation.

Bacterial cell wall components such as lipopolysaccharide (LPS) play important roles in the pathogenesis of various diseases. The presence of LPS in the body points to a bacterial infection that needs to be addressed by the immune system. Since LPS is a general component of Gram-negative bacteria, LPS constitutes a so called danger signal that can activate the immune system. LPS is recognized by the Toll-like receptor 4 (TLR4), a member of the larger family of Toll-like receptors involved in the recognition of varied danger signals or pathogen associated molecular patterns (PAMPs) associated with bacterial or viral infections. While recognition of LPS as danger signal is an important part of innate immunity, overstimulation or prolonged stimulation of the TLR4 receptor is connected to a variety of pathological conditions associated with chronic inflammation. Examples are various liver diseases such as alcoholic liver disease, nonalcoholic fatty liver disease, nonalcoholic steatohepatitis, chronic hepatitis B or C virus (HCV) infection, and HIV-HCV co-infection. Other diseases associated with TLR4 signaling are rheumatoid arthritis, artherosclerosis, psoriasis, Crohn's disease, uveitis, contact lens associated keratitis and corneal inflammation. In addition, TLR4-mediated signaling is involved in cancer progression and resistance to chemotherapy.

Immunoglobulins isotype E (IgE) are part of the adaptive immune system and as such involved in the protection against infections but also neoplastic transformation. IgE is bound by the high-affinity IgE receptor (FCER1) localized on mast cells and basophiles. Binding of IgE to FCER1 followed by cross-linking these complexes via specific antigens called allergens leads to the release of various factors from mast cells and basophils causing the allergic response. Among these factors are histamine, leukotrienes, various cytokines but also lysozyme, tryptase or β-hexosaminidase. The release of these factors is associated with allergic diseases such as allergic rhinitis, asthma, eczema and anaphylaxis.

Nuclear receptors are a protein superfamily of ligand-activated transcription factors. They are, unlike most other cellular receptors, soluble proteins localized to the cytosol or the nucleoplasm. Ligands for nuclear receptors are lipophilic molecules, among them steroid and thyroid hormones, fatty and bile acids, retinoic acid, vitamin D3 and prostaglandins (McEwan I. J., Methods in Molecular Biology: The Nuclear Receptor Superfamily, 505, 3-17). Upon ligand binding, nuclear receptors dimerize, thus triggering binding to specific transcription-factor-specific DNA response elements inside ligand-responsive gene promoters causing either activation or repression of gene expression. Given that nuclear receptors are responsible for mediating the activity of many broad-acting hormones such as steroids and important metabolites, the miss- and dysfunction of nuclear receptors is involved in the natural history of many diseases.

Using agonists or antagonists to modulate the activity of nuclear receptors is employed for therapeutic purposes. Modulation of glucocorticoid receptor (NR3C1) function using corticosteroids such as agonistic dexamethasone is common clinical practice for influencing inflammatory diseases. Another modulation of nuclear receptor activity is exemplified in oral contraception, wherein activation of the estrogen receptor (ESR1/ER) and the progesterone receptor is used to prevent egg fertilization in women. In another example, blocking the androgen receptor (AR) using anti-androgens such as flutamide or bicalutamide proved useful for the treatment of AR-dependent prostate cancers. Furthermore, blockage of the estrogen receptor by blocking estrogen synthesis and thus the availability of estrogen is a standard treatment for breast cancer in women or gynaecomastia in men.

Genetic mutations are at the heart of many inherited disorders. In general, such mutations can be classified into dominant or recessive regarding their mode of inheritance, with a dominant mutation being able to cause the disease phenotype even when only one gene copy—be it the maternal or the paternal—is affected, while for a recessive mutation to cause disease both, maternal and paternal, gene copies need to be mutated. Dominant mutations are able to cause disease by one of two general mechanisms, either by dominant-negative action or by haploinsufficiency. In case of a dominant-negative mutation, the gene product gains a new, abnormal function that is toxic and causes the disease phenotype. Examples are subunits of multimeric protein complexes that upon mutation prevent proper function of said protein complex. Diseases inherited in a dominant fashion can also be caused by haploinsufficiency, wherein the disease-causing mutation inactivates the affected gene, thus lowering the effective gene dose. Under these circumstances, the second, intact gene copy is unable to provide sufficient gene product for normal function. About 12'000 human genes are estimated to be haploinsufficient (Huang et al., 2010, PLoS Genet. 6(10), e1001154) with about 300 genes known to be associated with disease.

Neuronal survival critically depends on mitochondrial function with mitochondrial failure at the heart of many neurodegenerative disorders (Karbowski M., Neutzner A., 2012, Acta Neuropathol 123(2), 157-71). Besides their essential function in providing energy in form of ATP, mitochondria are critically involved in calcium buffering, diverse catabolic as well as metabolic processes and also programmed cell death. This important function of mitochondria is mirrored in the many cellular mechanisms in place to maintain mitochondria and to prevent mitochondrial failure and subsequently cell death (Neutzner A. et al., 2012, Semin Cell Dev Biol 23, 499-508). A central role among these processes plays the maintenance of a dynamic mitochondrial network with a balanced mitochondrial morphology. This is achieved by the so called mitochondrial morphogens that promote either fission of mitochondria in the case of Drp1, Fis1, Mff, MiD49 and MiD51—or fusion of mitochondrial tubules in the case of Mfn1, Mfn2 and OPA1. Balancing mitochondrial morphology is essential since loss of mitochondrial fusion is known to promote the loss of ATP production and sensitizes cells to apoptotic stimuli connecting this process to neuronal cell death associated with neurodegenerative disorders.

A key player in the process of mitochondrial fusion is optic atrophy 1 or OPA1. OPA1 is a large GTPase encoded by the OPA1 gene and essential for mitochondrial fusion. In addition, OPA1 plays an important role in maintaining internal, mitochondrial structure as component of the cristae. It was shown that downregulation of OPA1 gene expression causes mitochondrial fragmentation due to a loss of fusion and sensitizes cells to apoptotic stimuli. Mutations in OPA1 were identified to be responsible for about 70% of Kjer's optic neuropathy or autosomal dominant atrophy (ADOA). In most populations, ADOA is prevalent between 1/10'000 and 3/100'000 and is characterized by a slowly progressing decrease in vision starting in early childhood. The visual impairment ranges from mild to legally blind, is irreversible and is caused by the slow degeneration of the retinal ganglion cells (RGCs). In most cases, ADOA is non-syndromic, however, in about 15% of patients extra-ocular, neuro-muscular manifestations such as sensori-neural hearing loss are encountered. Until now, no viable treatment for this disease is available. Interestingly, certain OPA1 alleles were connected to normal tension, but not high tension glaucoma, highlighting again the importance of OPA1 for maintaining normal mitochondrial physiology.

### SUMMARY OF THE INVENTION

The invention relates to an artificial transcription factor comprising a polydactyl zinc finger protein targeting specifically a gene promoter fused to an inhibitory or activatory protein domain, a nuclear localization sequence, a protein transduction domain, and an endosome-specific protease recognition site, and to pharmaceutical compositions comprising such an artificial transcription factor. Furthermore the invention relates to the use of such artificial transcription factors for modulating the expression of genes, and in treating diseases wherein modulation of such gene expression is beneficial.

In a particular embodiment, the gene promoter targeted by the artificial transcription factors of the invention is a receptor gene promoter.

In another particular embodiment, the gene promoter targeted by the artificial transcription factors of the invention is a nuclear receptor gene promoter.

In another particular embodiment, the gene promoter targeted by the artificial transcription factors of the invention is a haploinsufficient gene promoter.

In a particular embodiment, the endosome-specific protease recognition site is a cathepsin recognition site, preferably a cathepsin B recognition site, for example the cathepsin B recognition site contained in the cathepsin B in vitro substrate prorenin (QPMKRLTLGN, SEQ ID NO: 28).

In another particular embodiment the invention relates to an artificial transcription factor variant comprising a polydactyl zinc finger protein targeting specifically a gene promoter fused to an inhibitory or activatory protein domain, a nuclear localization sequence, and an endosome-specific protease recognition site.

In a particular embodiment, the receptor gene promoter is the endothelin receptor A promoter (SEQ ID NO: 29). In another particular embodiment the invention relates to such an artificial transcription factor for use in influencing the cellular response to endothelin, for lowering or increasing endothelin receptor A levels, and for use in the treatment of diseases modulated by endothelin, in particular for use in the treatment of such eye diseases. Likewise the invention relates to a method of treating a disease modulated by endothelin comprising administering a therapeutically effective amount of an artificial transcription factor of the invention to a patient in need thereof.

In another particular embodiment, the receptor gene promoter is the endothelin receptor B promoter (SEQ ID NO: 30). In another particular embodiment the invention relates to such an artificial transcription factor for use in influencing the cellular response to endothelin, for lowering or increasing endothelin receptor B levels, and for use in the treatment of diseases modulated by endothelin, in particular for use in the treatment of such eye diseases. Likewise the invention relates to a method of treating a disease modulated by endothelin comprising administering a therapeutically effective amount of an artificial transcription factor of the invention to a patient in need thereof.

In another particular embodiment, the receptor gene promoter is the Toll-like receptor 4 promoter (SEQ ID NO: 31). In another particular embodiment the invention relates to such an artificial transcription factor for use in influencing the cellular response to lipopolysaccharide, for lowering or increasing Toll-like receptor 4 levels, and for use in the treatment of diseases modulated by lipopolysaccharide, in particular for use in the treatment of eye diseases. Likewise the invention relates to a method of treating a disease modulated by lipopolysaccharide comprising administering a therapeutically effective amount of an artificial transcription factor of the invention to a patient in need thereof.

In another particular embodiment, the receptor gene promoter is the high-affinity immunoglobulin epsilon receptor subunit alpha (FcER1A) promoter (SEQ ID NO: 32). In another particular embodiment the invention relates to such an artificial transcription factor for use in influencing the cellular response to immunoglobulin E (IgE), for lowering or increasing high-affinity IgE receptor levels, and for use in the treatment of diseases modulated by IgE, in particular for use in the treatment of eye diseases. Likewise the invention relates to a method of treating a disease modulated by IgE comprising administering a therapeutically effective amount of an artificial transcription factor of the invention to a patient in need thereof.

In another particular embodiment, the promoter region of the nuclear receptor gene is the glucocorticoid receptor promoter (SEQ ID NO: 33). In this particular embodiment the invention relates to an artificial transcription factor targeting the glucocorticoid receptor promoter for use in influencing the cellular response to glucocorticoids, for lowering or increasing glucocorticoid receptor levels, and for use in the treatment of diseases modulated by glucocorticoids, in particular for use in the treatment of eye diseases modulated by glucocorticoids. Likewise the invention relates to a method of treating a disease modulated by glucocorticoids comprising administering a therapeutically effective amount of an artificial transcription factor of the invention targeting the glucocorticoid receptor promoter to a patient in need thereof.

In another particular embodiment, the promoter region of the nuclear receptor gene is the androgen receptor promoter (SEQ ID NO: 34). In this particular embodiment the invention relates to an artificial transcription factor targeting the androgen receptor promoter for use in influencing the cellular response to testosterone, for lowering or increasing androgen receptor levels, and for use in the treatment of diseases modulated by testosterone. Likewise the invention relates to a method of treating a disease modulated by testosterone comprising administering a therapeutically effective amount of an artificial transcription factor of the invention targeting the androgen receptor promoter to a patient in need thereof.

In another particular embodiment, the promoter region of the nuclear receptor gene is the estrogen receptor promoter (SEQ ID NO: 35). In this particular embodiment the invention relates to such an artificial transcription factor targeting the estrogen receptor promoter for use in influencing the cellular response to estrogen, for lowering or increasing estrogen receptor levels, and for use in the treatment of diseases modulated by estrogen. Likewise the invention relates to a method of treating a disease modulated by estrogen comprising administering a therapeutically effective amount of an artificial transcription factor of the invention targeting the estrogen receptor promoter to a patient in need thereof.

Furthermore the invention relates to the use of such artificial transcription factors for increasing the expression from haploinsufficient gene promoters, and in treating diseases caused or influenced by such haploinsufficient gene promoters. Likewise the invention relates to a method of treating a disease caused or modulated by haploinsufficiency comprising administering a therapeutically effective amount of an artificial transcription factor of the invention targeting a haploinsufficient gene promoter to a patient in need thereof.

In a particular embodiment, the haploinsufficient gene promoter is the OPA1 promoter (SEQ ID NO: 36). In this particular embodiment the invention relates to an artificial transcription factor for use in enhancing the expression of the OPA1 gene, and for use in the treatment of diseases caused or modified by low OPA1 levels, in particular for use in the treatment of eye diseases. Likewise the invention relates to a method of treating a disease influenced by OPA1 comprising administering a therapeutically effective amount of an artificial transcription factor of the invention to a patient in need thereof.

The invention further relates to nucleic acids coding for an artificial transcription factor of the invention, vectors comprising these, and host cells comprising such vectors.

### BRIEF DESCRIPTION OF THE FIGURES

FIG. 1: Modulating Gene Expression Using Protease-Sensitive Transducible Artificial Transcription Factors

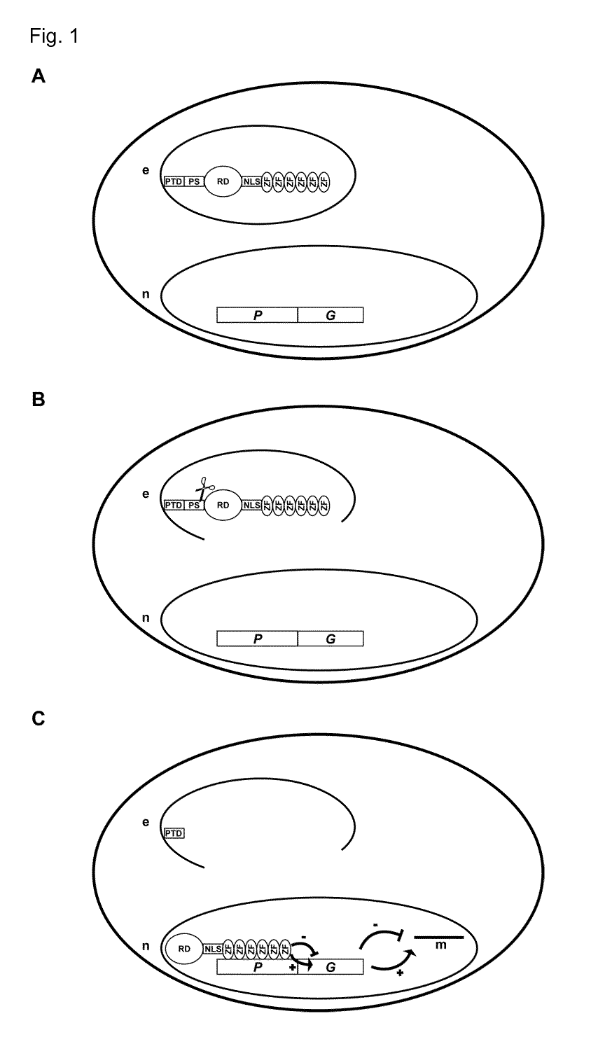

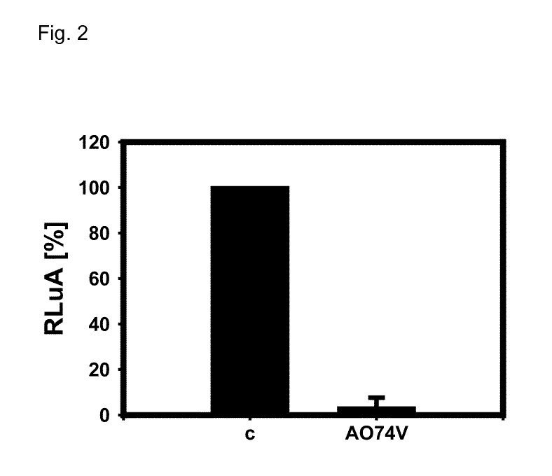

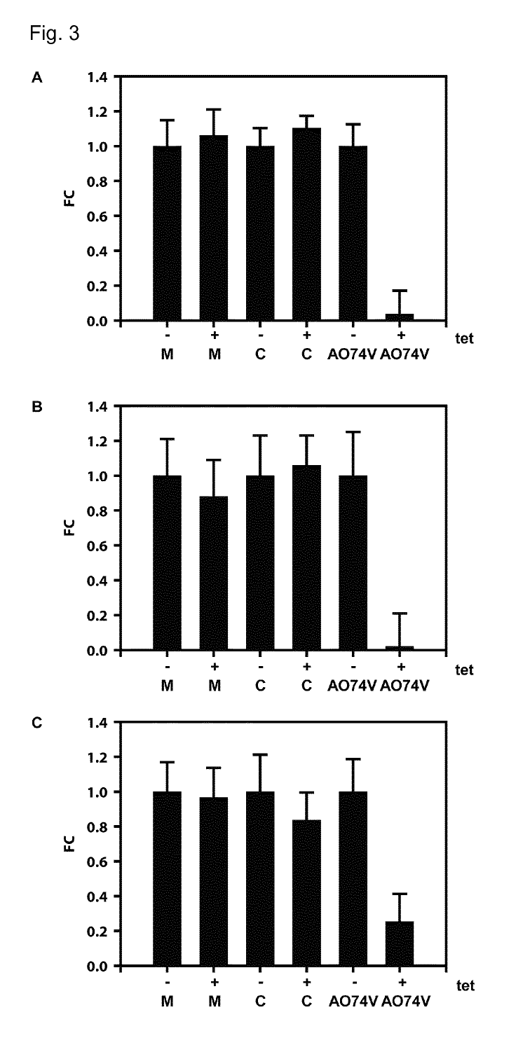

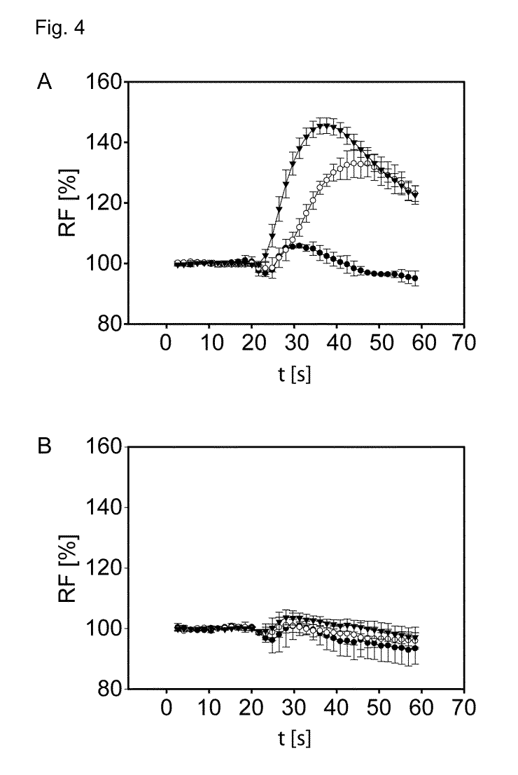

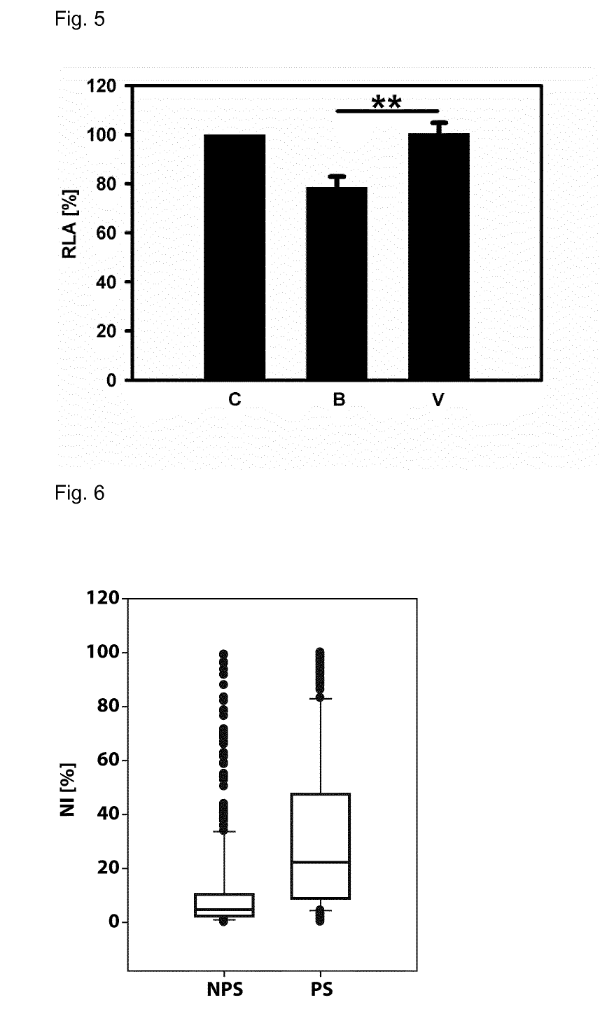

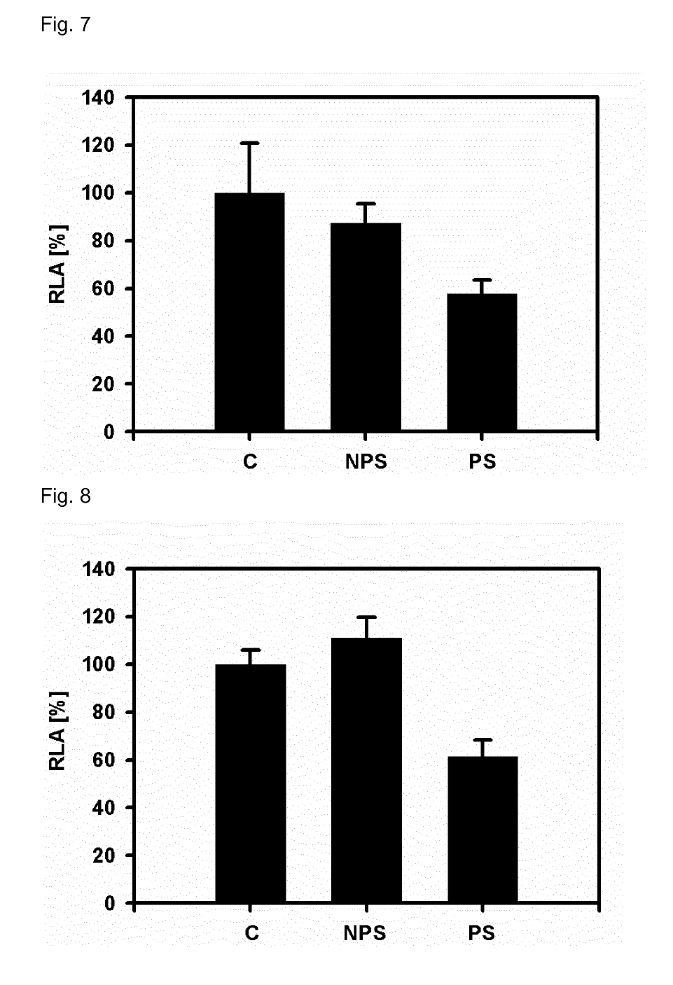

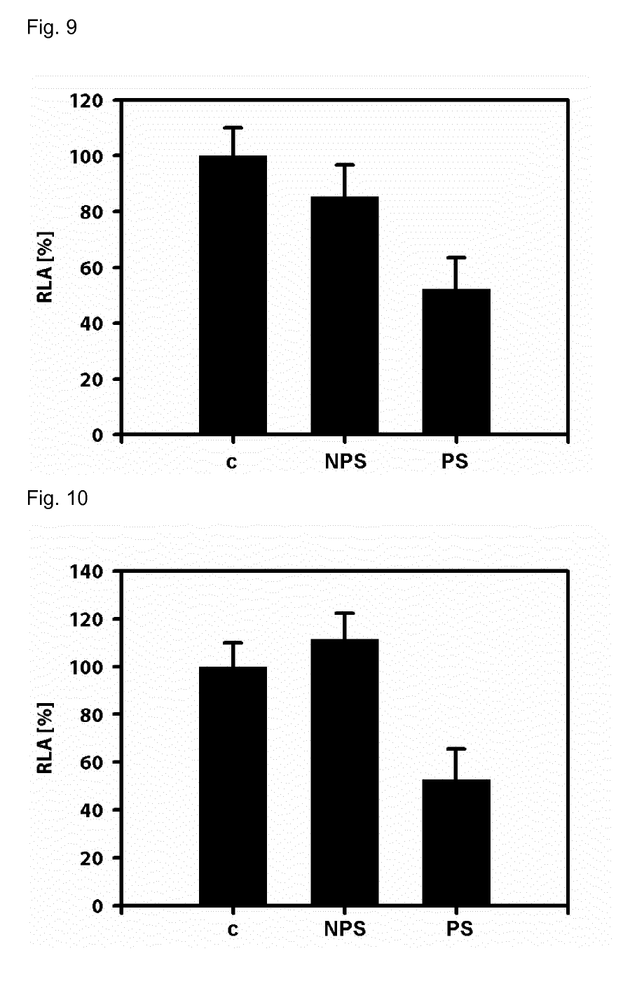

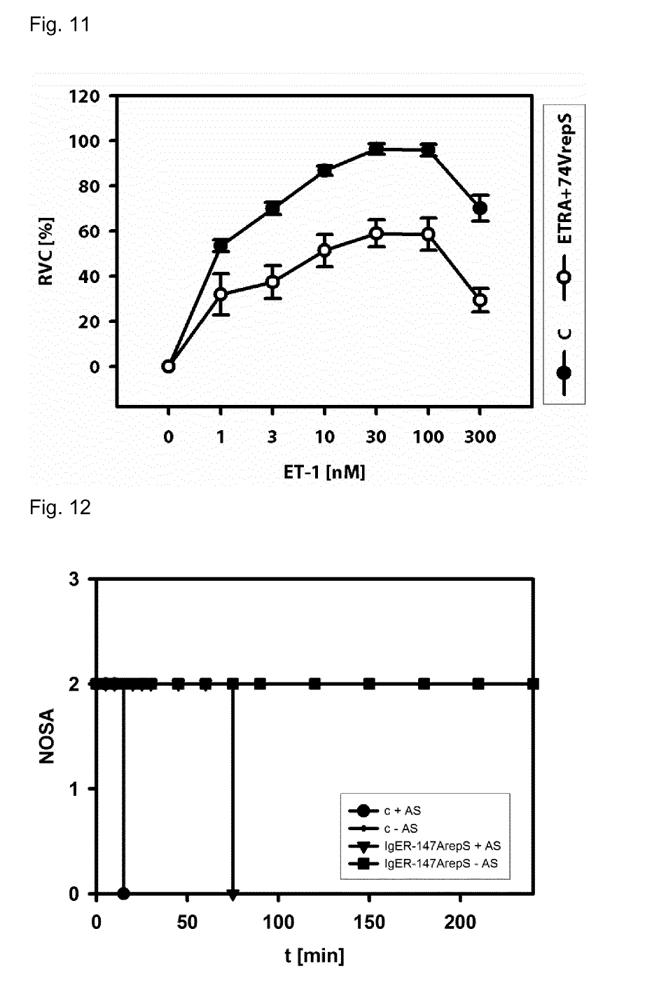

An artificial transcription factor comprising a protein transduction domain (PTD), an endosome-specific protease cleavage site (PS), a domain with transcription regulating activity (RD), a nuclear localization sequence (NLS), and a polydactyl zinc finger (ZF) protein specific for the promoter region (P) of a gene (G) enters the cell via an endocytotic mechanism. In FIG. 1A such an artificial transcription factor is trapped inside the endosomal compartment (e) unable to reach efficiently the nucleus (n). In FIG. 1B, an endosome-specific protease (symbolized by scissors) is activated during endosomal maturation, recognizes PS and cleaves the artificial transcription factor, thus separating PTD from RD-NLS-ZFₙ. Following rupture of the endosomal vesicle, the now cleaved artificial transcription factor is able to leave the endosomal compartment and is being transported to the nucleus, see FIG. 10. Upon binding to its target site in the promoter region P of gene G, production of mRNA (m) is either up- or downregulated (+ or −), depending on the transcription regulating activity of the regulatory domain RD.

FIG. 2: Activity of Artificial Transcription Factor Targeting ETRA

HeLa cells were co-transfected with an expression plasmid for AO74V, an ETRA-specific artificial transcription factor containing a SID domain, and a Gaussia luciferase/SEAP reporter plasmid containing the ETRA promoter (AO74V). Cells expressing YFP instead of AO74V served as control (c). 48 hours post transfection, luciferase activity was measured, normalized to SEAP activity and expressed as relative luciferase activity (RLuA) in percent of control.

FIG. 3: An ETRA-Specific Artificial Transcription Factor is Capable of Suppressing Expression of the Endogenous ETRA Gene
(A)+(B) HEK 293 FlpIn TRex cells stably expressing the ETRA-specific artificial transcription factor AO74V targeting ETRA_TS+74 (labeled AO74V) under the control of a tetracycline inducible promoter were treated with tetracycline (tet) for 24 hours or left untreated and ETRA mRNA levels were measured using quantitative RT-PCR. Cells containing stably integrated empty vector (labeled M) or an inactive version of AO74V lacking all cysteine residues involved in zinc complexation (labeled C) served as control. The expression construct was integrated into the FlpIn site (cells in panel A) present in these cells via homologous recombination or via TALEN-mediated double-strand repair into the AAVS1 safe harbor (cells in panel B).
   (C) HeLa cells containing tetracycline-inducible expression constructs for AO74V (labeled AO74V), inactive AO74V (labeled C) or empty vector control (labeled M) in the AAVS1 locus were induced with tetracycline (tet) for 24 hours or left untreated, and ETRA mRNA levels were quantified by RT-PCR. Shown are the average fold changes of three independent experiments of ETRA expression (FC) of tetracycline-induced cells relative to not induced cells. Error bars represent SD.

FIG. 4: ETRA-Specific Artificial Transcription Factor Blocks ET-1 Dependent Calcium Signaling

HEK 293 FlpIn TRex cells stably transfected with tetracycline-inducible expression construct for AO74V, an ETRA-specific artificial transcription factor containing a SID domain, were induced with 1 μg/ml tetracycline (B) or left uninduced (A) and treated with 0 (filled circles), 100 (empty circles), or 1000 (triangles) ng/ml ET-1. Calcium flux was measured and expressed as relative fluorescence (RF) in percent of base line vs. time (t) in seconds (s).

FIG. 5: ETRA-Specific Artificial Transcription Factor Blocks ET-1 Dependent Contraction of Human Uterine Smooth Muscle Cells

ETRA+74VrepSNPS blocks ET-1-dependent contraction of human uterine smooth muscle cells (hUtSMC). hUtSMC were embedded into 3-dimensional collagen lattices. C=cells treated with buffer as control. B=cells treated with buffer and ET-1. V=cells treated with ETRA+74VrepSNPS and ET-1. RLA=relative lattice area in % of control (C). Details are described below.

FIG. 6: Increased Endosomal Escape of ETRA+74VrepS Compared to ETRA+74VrepSNPS

HeLa cells were incubated for two hours in OptiMEM media with 1 μM cathepsin B-insensitive ETRA+74VrepSNPS (marked NPS) or cathepsin B-sensitive ETRA+74VrepS (marked PS) for 2 hours. Cells were fixed, stained using anti-myc epitope antibody to detect artificial transcription factors, and images were taken. Nuclear import (NI) of artificial transcription factor was determined using image analysis, and was expressed as percentage of maximal fluorescence signal. Shown is the average of three independent experiments with 200 cells/experiment.

FIG. 7: Inclusion of a Cathepsin B Recognition Site Increases Activity of an ETRA-Specific Artificial Transcription Factor in a Luciferase Reporter Assay

HEK 293 FlpIn cells stably expressing Gaussia luciferase under the control of a hybrid CMV/TS+74 (target site for ETRA+74VrepS/NPS) and secreted alkaline phosphatase under control of a constitutive CMV promoter were treated with ETRA+74VrepS (contains cathepsin site—labeled PS) or ETRA+74VrepSNPS (without cathepsin site—labeled NPS). Treatment with an inactive mutant of ETRA+74VrepS lacking all zinc complexing cystein residues was used as control (labeled C). Luciferase and secreted alkaline phosphatase activity was measured 24 hours after treatment. Luciferase activity was normalized to secreted alkaline phosphatase activity and expressed as percentage of control. Shown is the average of three independent experiments with three technical replicates. Statistical significance was analyzed using one-way ANOVA analysis with Tukey HSD posthoc test. Groups labeled C, NPS and PS are significantly different (P<0.05).

FIG. 8: Inclusion of a Cathepsin B Recognition Site Increases Activity of a TLR4-Specific Artificial Transcription Factor in a Luciferase Reporter Assay

HEK 293 FlpIn cells stably expressing Gaussia luciferase under the control of a hybrid CMV/TS-222 (target site for TLR4-222ArepS/NPS) and secreted alkaline phosphatase under control of a constitutive CMV promoter were treated with TLR4-222ArepS (contains cathepsin site—labeled PS) or TLR4-222ArepSNPS (without cathepsin site—labeled NPS). Treatment with an unrelated artificial transcription factor served as control (labeled C). Luciferase and secreted alkaline phosphatase activity were measured 24 hours after treatment. Luciferase activity was normalized to secreted alkaline phosphatase activity and expressed as percentage of control. Shown is the average of three independent experiments with three technical replicates. Error bars represent SD.

FIG. 9: Inclusion of a Cathepsin B Recognition Site Increases Activity of an AR-Specific Artificial Transcription Factor in a Luciferase Reporter Assay

HEK 293 FlpIn cells stably expressing Gaussia luciferase under the control of a hybrid CMV/TS-236 (target site for AR-236ArepS/NPS) and secreted alkaline phosphatase under control of a constitutive CMV promoter were treated with AR-236ArepS (contains cathepsin site—labeled PS) or AR-236ArepSNPS (without cathepsin site—labeled NPS). Treatment with an unrelated artificial transcription factor served as control (labeled C). Luciferase and secreted alkaline phosphatase activity were measured 24 hours after treatment. Luciferase activity was normalized to secreted alkaline phosphatase activity and expressed as percentage of control. Shown is the average of three independent experiments with three technical replicates. Error bars represent SD.

FIG. 10: Inclusion of a Cathepsin B Recognition Site Increases Activity of an FcER1A-Specific Artificial Transcription Factor in a Luciferase Reporter Assay

HEK 293 FlpIn cells stably expressing Gaussia luciferase under the control of a hybrid CMV/TS-147 (target site for IgER-147ArepS/NPS) and secreted alkaline phosphatase under control of a constitutive CMV promoter were treated with IgER-147ArepS (contains cathepsin site—labeled PS) or IgER-147ArepSNPS (without cathepsin site—labeled NPS). Treatment with an unrelated artificial transcription factor served as control (labeled C). Luciferase and secreted alkaline phosphatase activity were measured 24 hours after treatment. Luciferase activity was normalized to secreted alkaline phosphatase activity and expressed as percentage of control. Shown is the average of three independent experiments with three technical replicates. Error bars represent SD.

FIG. 11: Treatment with ETRA+74VrepS Decreases ET-1 Dependent Contraction of Human Coronary Vessels

Isolated human coronary vessel rings were incubated for 3 days with 1 μM of the ETRA-specific, cathepsin B-sensitive artificial transcription factor ETRA+74VrepS or buffer control. Vessel rings were then mounted into a wire myograph and vessel response to the vasoconstrictor U46619 as well to increasing concentrations of ET-1 was measured. The ET-1 response of the vessels was expressed as percentage of the U46619 response. Shown is the average of 8 vessels per condition from one human donor heart. Error bars represent SD.

FIG. 12: Treatment of Humanized NSG Mice with IgER-147ArepS Results in Delayed Death Following Induction of Anaphylactic Shock

Humanized NSG mice (NOD-scid IL2Rg^{null} implanted with human CD34⁺ cells) were treated with vehicle (labeled c) or IgER-147ArepS five days and two days before injection of anti-dinitrophenyl (anti-DNP) IgE antibodies. Injection of DNP-BSA (DNP coupled to bovine serum albumin) was used to induce anaphylaxis (labeled+AS), while injection of BSA served as control (labeled—AS). Shown is the number of surviving animals (NOSA) over time in minutes (labeled t [min]). Induction of anaphylaxis in vehicle treated mice (circle) leads to the rapid death of the animals (zero survivors at ten minutes after induction of anaphylaxis), while pretreatment with the FCER1A-specific artificial transcription factor IgER-147ArepS results in the prolonged survival of the treated animals.

### DETAILED DESCRIPTION OF THE INVENTION

The invention relates to an artificial transcription factor comprising a polydactyl zinc finger protein targeting specifically a gene promoter, for example a receptor gene promoter, in particular a membrane-bound receptor gene promoter or a nuclear receptor gene promoter, or a haploinsufficient gene promoter, fused to an inhibitory or activatory protein domain, a nuclear localization sequence, a protein transduction domain, and an endosome-specific protease recognition site, and to pharmaceutical compositions comprising such an artificial transcription factor. Furthermore the invention relates to the use of such artificial transcription factors for modulating the expression of genes, for example receptor genes, such as membrane-bound or nuclear receptor genes, or haploinsufficient genes, and in treating diseases caused or modulated by proteins encoded by the genes, the promoters of which are targeted by the transcription factors of the invention, for example receptor proteins, such as membrane-bound or nuclear receptor proteins, or proteins produced by haploinsufficient genes.

In the context of this invention, a promoter is defined as the regulatory region of a gene as well known in the art. Again in this context, a gene is defined, as well known in the art, as genomic region containing regulatory sequences as well as sequences for the gene product resulting in the production of proteins or RNAs.

In the context of the present invention, a polydactyl zinc finger protein targeting “specifically” a gene promoter means that the protein has a binding affinity of 20 nM or less towards its DNA target.

In the context of the present invention, a membrane-bound receptor gene causes the production of a protein or a protein that is part of a protein complex capable of binding to extracellular ligands and relaying the signal of ligand binding across the cellular membrane causing a cellular response. Also in the context of the present invention, a nuclear receptor gene causes the production of a soluble protein localized to the nucleus or the cytosol capable of binding cell-permeable ligands and capable of acting as transcription factor or accessory to a transcription factor for the modulation of gene expression upon binding their cognate ligand.

In the context of the present invention, a haploinsufficient gene is defined as a gene capable of causing the production of sufficient gene product in all cell types under all circumstances only if two functional gene copies are present in the genome. Thus, mutation of one gene copy of a haploinsufficient gene causes insufficient gene product generation in some or all cells of an organism under some or all physiological circumstances.

In the context of the invention, an endosome-specific protease recognition site is a peptide sequence that is recognized and cleaved in a sequence-specific manner by proteases resident in the endosomal compartment. Again in the context of this invention, a protein transduction domain is defined as a peptide capable of transporting proteins such as artificial transcription factors across the plasma membrane into the intracellular compartment.

Treatment of many diseases is based on modulating cellular receptor signaling. Examples are high blood pressure wherein beta blockers inhibit the function of the beta adrenergic receptors, depression wherein serotonin uptake blockers increase agonist concentration and thus serotonin receptor signaling, or glaucoma wherein prostaglandin analogues activate prostaglandin receptors, in turn decreasing intraocular pressure. Traditionally, small molecules either in the form of receptor agonist or antagonists are used to impact receptor signaling for therapeutic purposes. However, cellular receptor signaling can also be influenced by direct modulation of receptor protein expression.

Pathological processes amenable to direct modulation of receptor expression levels are, for example, the following: Patients with congestive heart failure due to congenital heart disease will benefit from the upregulation of beta-adrenoceptors, since downregulation of this receptor in the myocardium is associated with the risk of post-operative heart failure. In Parkinson's disease, treatment with dopaminergic medication suppresses the availability of dopamine receptors, thus, upregulation of dopamine receptor will improve the efficacy of dopaminergic medication. In epilepsy, insufficient expression of cannabinoid receptors in the hippocampus is involved in disease etiology, thus, upregulation of cannabinoid receptor will be a viable therapy for epileptic patients.

For genetic diseases caused by haploinsufficiency of a receptor protein, such as insulin-like growth factor I receptor causing growth retardation, but also others, additional activation of the remaining functional receptor gene will be beneficial for the patient. Furthermore and among others, induction and perpetuation of pathological autoimmunity is connected to inappropriate signaling from Toll-like receptors. Thus, downregulation of Toll-like receptors breaks the vicious cycle of various autoimmune diseases. In allergic disease, prevention of the IgE-mediated signaling through the high-affinity IgE receptor is useful to manage allergic reactions. In cancer, downregulation of growth factor receptors or upregulation of extracellular matrix receptors are beneficial for the prevention of tumor progression.

Among such receptor molecules are proteins from the so called seven-transmembrane or G protein coupled receptor (GPCR) family of proteins, characterized by seven transmembrane domains anchoring the receptor in the plasma membrane and a G protein dependent signaling cascade. Examples for such proteins are receptors A and B for endothelin. Other receptor proteins are anchored via a single transmembrane region, for example the receptor for lipopolysaccharide, Toll-like receptor 4, or various cytokine receptors, such as IL-4 receptor. Other receptors consist of multimeric protein complexes, for example the high-affinity receptor for IgE antibodies that consists of alpha, beta and gamma chains, or the T-cell receptor consisting of alpha, beta, gamma, delta, epsilon and zeta chains. Thus, subsumed under the term “receptor molecule” are proteins from different protein families with very different modes of action.

Receptors considered in the present invention are human receptor molecules encoded by HTR1A, HTR1B, HTR1D, HTR1E, HTR1F, HTR2A, HTR2B, HTR2C, HTR4, HTR5A, HTR5BP, HTR6, HTR7, CHRM1, CHRM2, CHRM3, CHRM4, CHRM5, ADORA1, ADORA2A, ADORA2B, ADORA3, ADRA1A, ADRA1B, ADRA1D, ADRA2A, ADRA2B, ADRA2C, ADRB1, ADRB2, ADRB3, AGTR1, AGTR2, APLNR, GPBAR1, NMBR, GRPR, BRS3, BDKRB1, BDKRB2, CNR1, CNR2, CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCKAR, CCKBR, C3AR1, C5AR1, GPR77, DRD1, DRD2, DRD3, DRD4, DRD5, EDNRA, EDNRB, GPER, FPR1, FPR2, FPR3, FFAR1, FFAR2, FFAR3, GPR42, GALR1, GALR2, GALR3, GHSR, FSHR, LHCGR, TSHR, GNRHR, GNRHR2, HRH1, HRH2, HRH3, HRH4, HCAR1, HCAR2, HCAR3, KISS1R, LTB4R, LTB4R2, CYSLTR1, CYSLTR2, OXER1, FPR2, LPAR1, LPAR2, LPAR3, LPAR4, LPAR5, S1PR1, S1PR2, S1PR3, S1PR4, S1PR5, MCHR1, MCHR2, MC1R, MC2R, MC3R, MC4R, MC5R, MTNR1A, MTNR1B, MLNR, NMUR1, NMUR2, NPFFR1, NPFFR2, NPSR1, NPBWR1, NPBWR2, NPY1R, NPY2R, PPYR1, NPY5R, NPY6R, NTSR1, NTSR2, OPRD1, OPRK1, OPRM1, OPRL1, HCRTR1, HCRTR2, P2RY1, P2RY2, P2RY4, P2RY6, P2RY11, P2RY12, P2RY13, P2RY14, QRFPR, PTAFR, PROKR1, PROKR2, PRLHR, PTGDR, PTGDR2, PTGER1, PTGER2, PTGER3, PTGER4, PTGFR, PTGIR, TBXA2R, F2R, F2RL1, F2RL2, F2RL3, RXFP1, RXFP2, RXFP3, RXFP4, SSTR1, SSTR2, SSTR3, SSTR4, SSTR5, TACR1, TACR2, TACR3, TRHR, TAAR1, UTS2R, AVPR1A, AVPR1B, AVPR2, OXTR, CCRL2, CMKLR1, GPR1, GPR3, GPR4, GPR6, GPR12, GPR15, GPR17, GPR18, GPR19, GPR20, GPR21, GPR22, GPR25, GPR26, GPR27, GPR31, GPR32, GPR33, GPR34, GPR35, GPR37, GPR37L1, GPR39, GPR42, GPR45, GPR50, GPR52, GPR55, GPR61, GPR62, GPR63, GPR65, GPR68, GPR75, GPR78, GPR79, GPR82, GPR83, GPR84, GPR85, GPR87, GPR88, GPR101, GPR119, O3FAR1, GPR132, GPR135, GPR139, GPR141, GPR142, GPR146, GPR148, GPR149, GPR150, GPR151, GPR152, GPR153, GPR160, GPR161, GPR162, GPR171, GPR173, GPR174, GPR176, GPR182, GPR183, LGR4, LGR5, LGR6, LPAR6, MAS1, MAS1L, MRGPRD, MRGPRE, MRGPRF, MRGPRG, MRGPRX1, MRGPRX2, MRGPRX3, MRGPRX4, OPN3, OPN5, OXGR1, P2RY8, P2RY10, SUCNR1, TAAR2, TAAR3, TAAR4P, TAAR5, TAAR6, TAAR8, TAAR9, CCBP2, CCRL1, DARC, CALCR, CALCRL, CRHR1, CRHR2, GHRHR, GIPR, GLP1R, GLP2R, GCGR, SCTR, PTH1R, PTH2R, ADCYAP1R1, VIPR1, VIPR2, BAI1, BAI2, BAI3, CD97, CELSR1, CELSR2, CELSR3, ELTD1, EMR1, EMR2, EMR3, EMR4P, GPR56, GPR64, GPR97, GPR98, GPR110, GPR111, GPR112, GPR113, GPR114, GPR115, GPR116, GPR123, GPR124, GPR125, GPR126, GPR128, GPR133, GPR144, GPR157, LPHN1, LPHN2, LPHN3, CASR, GPRC6A, GABBR1, GABBR2, GRM1, GRM2, GRM3, GRM4, GRM5, GRM6, GRM7, GRM8, GPR156, GPR158, GPR179, GPRC5A, GPRC5B, GPRC5C, GPRC5D, TAS1R1, TAS1R2, TAS1R3, FZD1, FZD2, FZD3, FZD4, FZD5, FZD6, FZD7, FZD8, FZD9, FZD10, SMO, GPR107, GPR137, OR51E1, TPRA1, GPR143, THRA, THRB, RARA, RARB, RARG, PPARA, PPARD, PPARG, NR1D1, NR1D2, RORA, RORB, RORC, NR1H4, NR1H5P, NR1H3, NR1H2, VDR, NR1I2, NR1I3, HNF4A, HNF4G, RXRA, RXRB, RXRG, NR2C1, NR2C2, NR2E1, NR2E3, NR2F1, NR2F2, NR2F6, ESR1, ESR2, ESRRA, ESRRB, ESRRG, AR, NR3C1, NR3C2, PGR, NR4A1, NR4A2, NR4A3, NR5A1, NR5A2, NR6A1, NR0B1, NR0B2, HTR3A, HTR3B, HTR3C, HTR3D, HTR3E, GABRA1, GABRA2, GABRA3, GABRA4, GABRA5, GABRA6, GABRB1, GABRB2, GABRB3, GABRG1, GABRG2, GABRG3, GABRD, GABRE, GABRQ, GABRP, GABRR1, GABRR2, GABRR3, GLRA1, GLRA2, GLRA3, GLRA4, GLRB, GRIA1, GRIA2, GRIA3, GRIA4, GRID1, GRID2, GRIK1, GRIK2, GRIK3, GRIK4, GRIK5, GRIN1, GRIN2A, GRIN2B, GRIN2C, GRIN2D, GRIN3A, GRIN3B, CHRNA1, CHRNA2, CHRNA3, CHRNA4, CHRNA5, CHRNA6, CHRNA7, CHRNA9, CHRNA10, CHRNB1, CHRNB2, CHRNB3, CHRNB4, CHRNG, CHRND, CHRNE, P2RX1, P2RX2, P2RX3, P2RX4, P2RX5, P2RX6, P2RX7, ZACN, AGER, TLR1, TLR2, TLR3, TLR4, TLR5, TLR6, TLR7, TLR8, TLR9, TLR10, TLR11, LILRA1, LILRA2, LILRA3, LILRA4, LILRA5, LILRA6, LILRB1, LILRB2, LILRB3a, LILRB4, LILRB5, LILRB6, LILRB7, EGFR, ERBB2, ERBB3, ERBB4, GFRa1, GFRa2, GFRa3, GFRa4, NPR1, NPR2, NPR3, NPR4, NGFR, NTRK1, NTRK2, NTRK3, EGFR, ERB2, ERB3, ERB4, INSR, IRR, IG1R, PDGFalpha, PDGFbeta, Fms, Kit, Flt3, FGFR1, FGFR2, FGFR3, FGFR4, BFR2, VGR1, VGR2, VGR3, EPA1, EPA2, EPA3, EPA4, EPA5, EPA7, EPA8, EPB1, EPB2, EPB3, EPB4, EPB6, TrkA, TrkB, TrkC, UFO, TYRO3, MERK, TIE1, TIE2, RON, MET, DDR1, DDR2, RET, ROS, LTK, ROR1, ROR2, RYK, PTK7, and KIT.

Further receptors considered are human receptors recognizing interleukin (IL)-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, leptin, interferon-alpha, interferon-beta, interferon-gamma, tumor necrosis factor alpha, lymphotoxin, prolactin, oncostatin M, leukemia inhibitory factor, colony-stimulating factor, immunoglobulin A, immunoglobulin D, immunoglobulin G, immunoglobulin M, immunoglobulin E, human leukocyte antigen (HLA) A, HLA-B, HLA-C, HLA-E, HLA-F, HLA-G, HLA-DP, HLA-DQ, HLA-DR, transforming growth factor alpha, transforming growth factor beta, nerve growth factor, brain-derived neurotrophic factor, neurotrophin-3, neurotrophin-4, adrenomedullin, angiopoietin, autocrine motility factor, bone morphogenetic proteins, erythropoietin, fibroblast growth factor, glial cell line-derived neurotrophic factor, granulocyte colony-stimulating factor, granulocyte macrophage colony-stimulating factor, growth differentiation factor-9, hepatocyte growth factor, hepatoma-derived growth factor, insulin-like growth factor, insulin, migration-stimulating factor, myostatin, platelet-derived growth factor, thrombopoietin, vascular endothelial growth factor, placental growth factor, connective tissue growth factor, and growth hormone.

Further considered are receptors encoded by homologous non-human genes, for example by porcine, equine, bovine, feline, canine, or murine genes; and receptors encoded by homologous plant receptor genes, for example genes found in crop plants such as wheat, barley, corn, rice, rye, oat, soybean, peanut, sunflower, safflower, flax, beans, tobacco, or life-stock feed grasses, and genes found in fruit plants such as apple, pear, banana, citrus fruit, grape or the like.

In contrast to almost all other cellular receptors that are membrane-anchored and consist or contain membrane-spanning proteins, nuclear receptors are soluble proteins incorporating ligand binding and transcription factor activity in one polypeptide. Nuclear receptors are either localized in the cytosol or the nucleoplasm, where they are activated upon ligand binding, dimerize and become active transcription factors regulating a vast array of transcriptional programs. Unlike above mentioned membrane-anchored receptors that bind their ligands outside the cell and transduce the signal across the plasma membrane into the cell, nuclear receptors bind lipophilic ligands that are capable of crossing the plasma membrane to gain access to their cognate receptor. In addition, most receptors rely on intricate signal amplification mechanisms before the intended cellular outcome is achieved. Nuclear receptors, on the other hand, directly convert the binding of a ligand into a cellular response.

Treatment of many diseases is based on modulating nuclear receptor signaling. Examples are inflammatory processes, wherein glucocorticoids activate the glucocorticosteriod receptor, prostate cancer, wherein antagonists of androgen receptor possess beneficial therapeutic effect, or breast cancer, wherein blocking estrogen receptor signaling proves useful. Traditionally, small molecules either in the form of nuclear receptor agonist or antagonists are used to impact receptor signaling for therapeutic purposes. However, nuclear receptor signaling can also be influenced by direct modulation of nuclear receptor protein expression, and such modulation is the subject of the present invention.

Nuclear receptors considered in the present invention are human nuclear receptors encoded by the human genes AR, ESR1, ESR2, ESRRA, ESRRB, ESRRG, HNF4A, HNF4G, NR0B1, NR0B2, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, PGR, PPARA, PPARD, PPARG, RARA, RARB, RARG, RORA, RORB, RORC, RXRA, RXRB, RXRG, THRA, THRB and VDR.

Further considered are non-human nuclear receptors, for example porcine, equine, bovine, feline, canine, or murine transcription factors, encoded by genes related to the mentioned human nuclear receptor genes.

For genetic diseases caused by haploinsufficiency of a gene promoter, such as insulin-like growth factor I receptor haploinsufficiency causing growth retardation or OPA1 haploinsufficiency causing dominant optic atrophy, but also others, additional activation of the remaining functional gene copy is beneficial for the patient. Artificial transcription factors of the invention are capable of increasing expression from haploinsufficient gene promoters, thus suitable for the treatment of diseases associated with haploinsufficiency.

Considered in the present invention are the following human genes and their respective promoters associated with haploinsufficiency, and disease as amenable to treatment using artificial transcription factors of the invention: PRKAR1A, FBN1, ELN, TCOF1, ENG, GLI3, TCF4, GRN, NKX2-1, SOX10, SHOX, MC4R, GATA3, NKX2-5, TBX1, COL10A1, PAX6, LMX1B, BMPR2, PAX9, SOX9, TRPV4, SPAST, TBX5, TWIST1, EHMT1, FOXC2, TBX3, TNXB, DSP, OPA1, TRPS1, RUNX2, SCN1A, HOXD13, NSD1, SATB2, PRPF31, SOX2, COL6A1, APC, RAI1, PAX3, ZEB2, SLC40A1, AFG3L2, KCNQ2, SALL1, PPARG, GDF5, GCH1, MYH9, SALL4, PITX2, FOXF1, RAD51, PKD2, NFKBIA, MSX1, MSX2, COL3A1, SH3TC2, SBDS, SIX6, KRIT1, SLC33A1, PARK2, ABCA4, MYOC, PAFAH1B1, CDKN1C, CREBBP, FGF3, MYF6, MPZ, ITPR1, EDN3, C3, TYRP1, OFC12, ATM, FOXP2, PHOX2B, COCH, PITX1, EYA1, FOXC1, KLF1, GATA4, KIT, MYCN, COL5A1, RNF135, MIR146A, SI, NLRP12, NDUFA13, SPRED1, REEP1, SLC6A19, CHD7, NCF1, IRF6, RXFP2, ZMPSTE24, ATL 1, EGLN1, NLRP3, KIF1B, BCMO1, SLC6A20, FOXL2, RTN4R, TSC1, WWOX, POLG2, LGI1, RECQL3, CNTNAP2, ATP2C1, KCNQ4, RPS19, ABCC6, STXBP1, NBN, ROBO1, ROR2, AGRP, STK11, KCNJ10, LHX4, FGF10, LIG4, ACVRL1, CAV3, GDF6, SMAD4, MYBPC3, IRS2, MSH6, ABCC8, GARS, CDKN2A, PORCN, PHEX, ARX, DMD, TPM1, NOTCH1, ABL1, RYR1, PTH1R, PAX8, PAX2, BRAF, MAPT, MC3R, KCNH2, LMNA, KRT5, SOD1, IGF1, MNX1, HNF1A, SLC2A1, GCK, GABRG2, FUS, DSG2, DCC, OFC1, CHRNA4, BRCA1, BDNF, BMP2, ATP2A2, ALX4, MITF, SIX3, SMARCB1, RANBP2, GDNF, MYC, ATP1A2, SLC6A4, FOXG1, IGF1R, FGFR1 and SERPINA6.

Further considered are non-human genes, for example porcine, equine, bovine, feline, canine, or murine genes, as well as their homologous human genes, plant genes, for example genes found in crop plants such as wheat, barley, corn, rice, rye, oat, soybean, peanut, sunflower, safflower, flax, beans, tobacco, or life-stock feed grasses, and genes found in fruit plants such as apple, pear, banana, citrus fruit, grape or the like, under the control of a haploinsufficient promoter.

Artificial transcription factors are useful for modulating gene expression, and thus are useful for the treatment of diseases wherein the modulation of gene expression is beneficial. While conventional drugs modulate the activity of a certain protein, e.g. by agonistic or antagonistic action, artificial transcription factors alter the availability of these proteins either by increasing or decreasing gene expression.

Using the traditional small molecule approach, the identification of therapeutically active small molecules acting through modulation of protein activity mostly relies on extensive and time-consuming screening procedures among a wide variety of different molecules from different classes of substances, and modulation of gene expression by small molecules is so far not possible. In contrast, artificial transcription factors of the invention all belong to the same substance class with a highly defined overall composition. Two hexameric zinc finger protein-based artificial transcription factors targeting two very diverse promoter sequences still have a minimal amino acid sequence identity of 85% with an overall similar tertiary structure, and can be generated via a standardized method (as described below) in a fast and economical manner. Thus, artificial transcription factors of the invention combine, in one class of molecule, exceptionally high specificity for a very wide and diverse set of targets with overall similar composition. As for all biologicals, immunogenicity in the form of anti-drug antibodies and the associated immunological reaction are a concern. However, due to the high conservation of zinc finger modules, such an immunological reaction will be minor or absent following application of artificial transcription factors of the invention, or might be avoided or further minimized by small changes to the overall structure eliminating immunogenicity while still retaining target site binding and thus function. Furthermore, modification of artificial transcription factors of the invention with polyethylene glycol is considered to reduce immunogenicity.

Since artificial transcription factors are tailored to act specifically on the promoter region of specific genes, the use of artificial transcription factors allows for selectively targeting even closely related proteins. This is based on the only loose conservation of the promoter regions even of closely related proteins. Taking advantage of the high selectivity of the artificial transcription factors according to the invention, even a tissue-specific targeting of a drug action is possible based on the oftentimes tissue-specific expression of certain members of a given protein family that are individually addressable using artificial transcription factors.

In addition, formulation of artificial transcription factors into drugs can rely on previous experience further expediting the drug development process.

However, artificial transcription factors need to be present in the nuclear compartment of cells in order to be effective as they act through modulation of gene expression. Until now, the method of choice for the therapeutic delivery of artificial transcription factors is either in the form of plasmid DNA through transfection or by employing viral vectors. Plasmid transfection for therapeutic purposes has low efficacy, while viral vectors have exceptionally high potential for immunogenicity, thus limiting their use in repeated application of a certain treatment. Thus other modes of delivering artificial transcription factors, for example in protein form instead of as nucleic acids, are required.

Protein transduction domain (PTD) mediated, intracellular delivery of artificial transcription factors is a new way of taking advantage of the high selectivity and versatility of artificial transcription factors in a novel fashion. Protein transduction domains are small peptides capable of crossing the plasma membrane barrier and delivering cargo proteins into the cell. Such protein transduction domains are, for example, the HIV derived TAT peptide, mT02, mT03, R9, ANTP, and others. The mode of cellular uptake is likely by endocytosis, and it was shown that the TAT peptide is able to induce a cell-type independent macropinocytotic uptake when fused to cargo proteins (Wadia J. S. et al., 2004, Nat Med 10, 310-315). While crossing the barrier of the plasma membrane and uptake into endosomal vesicles is the first step in entering the cell, topologically, the inside of the endosomal compartment is identical to the outside of the cell. Thus, endosomal localization is not equivalent to cytoplasmic or nucleoplasmic localization. However, likely through leakiness of the endosomal compartment and/or some intrinsic property of the cargo or the protein transduction domain in terms of modulating membrane integrity, delivered proteins are capable to escape endosomes and reach other truly intracellular targets. The co-delivery of the membrane-active, fusogenic peptide TAT-HA2 or others such as GALA or KALA peptide improved endosomal escape of delivered proteins somewhat due to the disintegration of endosomal vesicles. Indeed, mechanisms capable of disrupting the endosomal membrane are the state-of-the-art for increased endosomal escape of cargo proteins delivered using a protein transduction domain.

However, membrane disrupting agents are not as efficient in promoting delivery as expected. This might be due to the inherent properties of protein transduction domains. Protein transduction domains are known to strongly interact with cellular membranes. This strong membrane interaction is part of the mechanism by which protein internalization and protein delivery is triggered. Thus, following internalization into endosomes, this strong membrane interaction of the protein transduction domain now with the inside of the endosomal membrane might actually inhibit redistribution even after the rupture of endosomal vesicles. TAT-fused artificial transcription factors may mainly reside in the endosomal compartment with some nuclear localization. Interestingly, in a large percentage of cells stained for TAT-artificial transcription factor, ruptured endosomal vesicles were found open to the cytosol with endosomal membranes clearly decorated with TAT fusion protein, consistent with endosomal entrapment of a considerable amount of delivered protein even after endosomal membrane rupture. Thus, while essential for uptake into the cell, protein transduction domains hinder efficient subcellular localization once protein transduction takes place.

The endosome is a very dynamic organelle known to mature and acquire lysosomal characteristics, such as acquiring proteases and indicating a drop in vesicular pH before fusion with the lysosomal compartment and proteolytic degradation of the endosomal content. The process of endosomal maturation accompanied by an increase in lumenal proteolytic activity is detrimental for therapeutic proteins delivered using protein transduction domains, since such proteins are then subject to proteolysis. However, this process can be turned into an advantage. Endosomal maturation is a sequential process wherein different sets of proteases are activated at different stages in a pH-dependent manner. Interestingly, proteases activated early in the process involved in protein processing are more sequence specific than proteases activated late during maturation essential for general hydrolysis of proteins. Now, incorporation of a cleavage site for an early endosomal protease between the protein transduction domain and the cargo protein leads to the sequence-specific digestion of the therapeutic protein separating the protein transduction domain from the cargo protein once the therapeutic protein has reached the endosomal lumen. Thus, upon endosomal rupture, frequently observable following TAT-mediated delivery of artificial transcription factors, the cargo protein is no longer bound to the inside of the endosomal membrane due to inherent properties of the protein transduction domain but is detached from the membrane in order to escape into the cytosol (FIG. 1).

Proteases active in the endosomal compartment are the cathepsins, a large family of diverse proteases with different characteristics in terms of pH optimum and sequence specificity. Cathepsin B, for example, has a pH optimum around neutral pH and is sequence specific making this protease a good choice as TAT-cargo fusion protein processing endosomal protease. However, other cathepsins, such as cathepsin H, L, S, C, K, O, F, V, X, W, D or E, might also be useful for the purpose of separating protein transduction domains from their cargo once the endosomal compartment is reached. Taking advantage of the tissue and cell-type specific expression of certain cathepsins, improved subcellular localization and thus effective therapeutic action of such therapeutics can be limited to certain cell types by including cathepsin recognition sites specific for these tissues or cells.

The use of a protease recognition site, such as a cathepsin B site, in the present invention to improve the endosomal escape of cargo proteins such as artificial transcription factors, is beyond state-of-the-art. Unlike known approaches, no additional endosomal vesicle rupture is introduced, but the cargo protein is separated from the protein transduction domain after entry into the endosome to allow for efficient escape from the endosome following base-line vesicle rupture.

In known examples, cell penetrating peptides were used together with protease recognition sites (EP 2 399 939, WO 2008/063113), for the sole purpose of increasing the selectivity of protein transduction. By masking the protein transduction domain with an inhibitory peptide, cargo transport across the plasma membrane is prevented. Upon encountering a tissue and/or cell type-specific extracellular protease this inhibitory peptide is cleaved allowing now for protein transport across the plasma membrane. These state-of-the art examples are substantially different from the particular constructs leading to increase of endosomal escape described in the present invention.

In another known example, an endosomal protease recognition site was used together with a protein transduction domain (WO 2005/003315). In this instance, the procedure provided is a method of transport of DNA (used for transfection) into cells. The endosomal protease site was only used as a marker to confirm entry of the DNA complex via an endosomal route, but not to enhance endosomal escape of DNA.

In contrast to this described use of an endosomal protease recognition site as a marker, the constructs of the present invention provide increased endosomal escape of a functional protein, not a marker for the detection of a route of entry of a DNA complex.

Further, the artificial transcription factors of the invention comprise a nuclear localization sequence (NLS). Nuclear localization sequences considered are amino acid motifs conferring nuclear import through binding to proteins defined by gene ontology GO:0008139, for example clusters of basic amino acids containing a lysine residue (K) followed by a lysine (K) or arginine (R) residue, followed by any amino acid (X), followed by a lysine or arginine residue (K-K/R-X-K/R consensus sequence, Chelsky D. et al., 1989 Mol Cell Biol 9, 2487-2492) or the SV40 NLS (SEQ ID NO: 37), with the SV40 NLS being preferred.

The artificial transcription factor of the present invention might also contain other transcriptionally active protein domains of proteins defined by gene ontology GO:0001071 such as N-terminal KRAB, C-terminal KRAB, SID and ERD domains, preferably KRAB or SID. Activatory protein domains considered are the transcriptionally active domains of proteins defined by gene ontology GO:0001071, such as VP16, VP64 (tetrameric repeat of VP16), CJ7, p65-TA1, SAD, NF-1, AP-2, SP1-A, SP1-B, Oct-1, Oct-2, Oct2-5x, MTF-1, BTEB-2, and LKLF, preferably VP64 and AP-2.

Considered are also artificial transcription factors of the invention containing pentameric, hexameric, heptameric or octameric zinc finger proteins wherein individual zinc finger modules are exchanged to improve binding affinity towards target sites of the respective nuclear receptor promoter gene or to alter the immunological profile of the zinc finger protein for improved tolerability.

The domains of the artificial transcription factors of the invention may be connected by short flexible linkers. A short flexible linker has 2 to 8 amino acids, preferably glycine and serine. A particular linker considered is GGSGGS (SEQ ID NO: 38). Artificial transcription factors may further contain markers, such as epitope tags, to ease their detection and processing.

Co-delivery of fusogenic peptides, such as TAT-HA2, GALA or KALA, was shown to increase endosomal escape of cargo proteins following protein transduction. However, co-delivery of such peptides is probably not a viable option to increase protein delivery in vivo, as this implies a two-component system—fusogenic peptide and therapeutic protein—with likely differences in distribution and elimination behavior for the components in a living system.

Incorporation of fusogenic peptides into the therapeutic protein is a better option to circumvent this two-component problem mentioned above. However, these fusogenic peptides have certain restrictions in terms of size, in possibility to interact, and in N- as well as C-terminal amino acid sequence in order to act as fusogen for endosomal membranes. Thus, simply incorporating a fusogenic peptides into a cargo protein is not yet a viable option to increase endosomal escape.

However, incorporation of fusogenic peptides into artificial transcription factors of the invention via an endosomal protease-sensitive linker region allows for the simultaneous delivery of cargo protein and fusogenic peptide into the endosomal lumen. Once inside the endosome, separation of the artificial transcription factor from the protein transduction domain occurs, and in addition the liberation of fusogenic peptides. Through the inclusion of multiple repeats of fusogenic peptides, separating each fusogenic peptide subunit by an endosomal protease site, multiple fusogenic peptides are delivered to the endosome, thereby increasing endosomal rupture.

Selection of Target Sites within a Given Promoter Region

Target site selection is crucial for the successful generation of a functional artificial transcription factor. For an artificial transcription factor to modulate target gene expression in vivo, it must bind its target site in the genomic context of the target gene. This necessitates the accessibility of the DNA target site, meaning chromosomal DNA in this region is not tightly packed around histones into nucleosomes and no DNA modifications such as methylation interfere with artificial transcription factor binding. While large parts of the human genome are tightly packed and transcriptionally inactive, the immediate vicinity of the transcriptional start site (−1000 to +200 bp) of an actively transcribed gene must be accessible for endogenous transcription factors and the transcription machinery, such as RNA polymerases. Thus, selecting a target site in this area of any given target gene will greatly enhance the success rate for the generation of an artificial transcription factor with the desired function in vivo.

Selection of Target Sites within the Human Endothelin Receptor A (ETRA) Promoter Region

The promoter region of the human ETRA gene was analyzed for the presence of potential 18 bp target sites with the general composition of (G/CANN)₆, wherein G is the nucleotide guanine, C the nucleotide cytosine, A the nucleotide adenine and N stands for each of the four nucleotide guanine, cytosine, adenine and thymine. Three target sites were selected based on their position relative to the transcription start site and designated ETRA_TS-37 (SEQ ID NO: 39), ETRA_TS-50 (SEQ ID NO: 40) and ETRA_TS+74 (SEQ ID NO: 41). Considered are also target sites of the general composition (G/C/ANN)₅ and (G/C/ANN)₆ chosen from the regulatory region of the ETRA gene 2000 bp upstream of the transcription start.

Selection of Target Sites within the Human Endothelin Receptor B (ETRB) Promoter Region

The promoter region of the human ETRB gene was analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. Two target sites were selected based on their position relative to the transcription start site and designated ETRB_TS-1149 (SEQ ID NO: 42) and ETRB_TS-487 (SEQ ID NO: 43). Considered are also target sites of the general composition (G/C/ANN)₅ and (G/C/ANN)₆ chosen from the regulatory region of the ETRB gene 2000 bp upstream of the transcription start.

Selection of Target Sites within the Human Toll-Like Receptor 4 (TLR4) Promoter Region

The promoter region of the human TLR4 gene was analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. Two target sites were selected based on their position relative to the transcription start site and designated TLR4_TS-55 (SEQ ID NO: 44), TLR4_TS-222 (SEQ ID NO: 45) and TLR4TS-276 (SEQ ID NO: 46). Considered are also target sites of the general composition (G/C/ANN)₅ and (G/C/ANN)₆ chosen from the regulatory region of the TLR4 gene 2000 bp upstream of the transcription start.

Selection of Target Sites within the Human High-Affinity IgE Receptor A (FCER1A) Promoter Region

The promoter region comprising the transcriptional start site of the human FCER1A gene was analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. Two target sites were selected based on their position relative to the transcription start site and designated IgER_TS-147 (SEQ ID NO: 47) and IgER_TS17 (SEQ ID NO: 48). Considered are also target sites of the general composition (G/C/ANN)₅ and (G/C/ANN)₆ chosen from the regulatory region of the FCER1A gene 2000 bp upstream of the transcription start.

Selection of Target Sites within the Human TGFbR1 Gene

The promoter region comprising the transcriptional start site of the human TGFbR1 gene was analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. One target site was selected based on its position relative to the translation start site and designated TGF_TS-390 (SEQ ID NO: 49). Considered are also target sites of the general composition (G/C/ANN)₅ and (G/C/ANN)₆ chosen from the regulatory region of the TGFbR1 gene 2000 bp upstream of the transcription start.

Selection of Target Sites within the Human Glucocorticoid, Androgen and Estrogen Receptor Gene Promoters

The promoter regions comprising 1000 bp including the transcriptional start site of the human glucocorticoid, androgen and estrogen receptor gene were analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. Three to four target sites in each promoter were selected based on their position relative to the transcription start site. The target sites found in the glucocorticoid receptor gene promoter are GR_TS1 (SEQ ID NO: 50), GR_TΩ (SEQ ID NO: 51), GR_TS3 (SEQ ID NO: 52), the target sites for the androgen receptor are AR_TS1 (SEQ ID NO: 53), AR_TS2 (SEQ ID NO: 54), AR_TS3 (SEQ ID NO: 55) and AR_TS-236 (SEQ ID NO: 56). The target sites identified in the estrogen receptor gene promoter are ER_TS1 (SEQ ID NO: 57), ER_TS2 (SEQ ID NO: 58) and ER_TS3 (SEQ ID NO: 59). Considered are also target sites of the general composition (G/C/ANN)₅ and (G/C/ANN)₆ chosen from the regulatory region of the glucocorticoid receptor, the estrogen receptor and the androgen receptor 2000 bp upstream of the transcription start.

Selection of Target Sites within the Human OPA1 Gene Promoter

A region 1000 bp upstream of the start codon of the human OPA1 open reading frame was analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. Four target sites, OPA_TS1 (SEQ ID NO: 60), OPA_TS2 (SEQ ID NO: 61), OPA_TS3 (SEQ ID NO: 62), and OPA_TS-165 (SEQ ID NO: 63) were chosen. Further considered are also target sites of the general composition (G/C/ANN)₅ and (G/C/ANN)₆ chosen from the regulatory region of the OPA1 open reading frame.

### Artificial Transcription Factors Targeting Receptor Gene Promoters

Hexameric zinc finger proteins targeting specific gene target sites were selected using a modified yeast one hybrid screen. Yeast containing the aureobasidin A resistance gene under control of a chimeric yeast promoter comprising a target site and the yeast cyc1 minimal promoter were transformed with a plasmid library of expression plasmids for hybrid activating transcription factors consisting of hexameric zinc finger proteins fused to the GAL4 activation domain. Upon binding of such a hybrid transcription factor to the chimeric yeast promoter described above, the aureobasidin A resistance gene is transcribed and confers resistance to this antibiotic relative to the strength of the interaction between the hexameric zinc finger and the target site tested. Using increasing selection pressure, hexameric zinc finger proteins with strong binding affinity to specific target sites are selected. Such zinc finger proteins specifically targeting are fused to the protein transduction domain TAT as well as the transcription activating domain VP64 or the inhibitor domains N-KRAB, C-KRAB or SID to obtain artificial transcription factors. To generate cathepsin B sensitive artificial transcription factors a cathepsin B site is introduced between the TAT protein transduction domain and the artificial transcription factor consisting of nuclear localization sequence, zinc finger protein and regulatory domain.

ETRA-specific hexameric zinc fingers are ETRA-37B (SEQ ID NO: 64), ETRA-37D (SEQ ID NO: 65), ETRA-50A (SEQ ID NO: 66), ETRA-50B (SEQ ID NO: 67), ETRA-50C (SEQ ID NO: 68), ETRA-50D (SEQ ID NO: 69), ETRA-50E (SEQ ID NO: 70), ETRA-50F (SEQ ID NO: 71), ETRA-50G (SEQ ID NO: 72), ETRA-50H (SEQ ID NO: 73), ETRA-501 (SEQ ID NO: 74), ETRA-50J (SEQ ID NO: 75), ETRA-50K (SEQ ID NO: 76), ETRA-50L (SEQ ID NO: 77), ETRA-50M (SEQ ID NO: 78), ETRA+74E (SEQ ID NO: 79), ETRA+74V (SEQ ID NO: 80), ETRA+74R (SEQ ID NO: 81), ETRA+74AA (SEQ ID NO: 82), ETRA+74AB (SEQ ID NO: 83). ETRA+74AC (SEQ ID NO: 84). ETRA+74AD (SEQ ID NO: 85). ETRA+74AE (SEQ ID NO: 86). ETRA+74AF (SEQ ID NO: 87). ETRA+74AG (SEQ ID NO: 88), and ETRA+74AH (SEQ ID NO: 89). Resulting ETRA-specific cathepsin B-sensitive VP64-(akt) or SID- (repS) containing transcription factors are ETRA+74Eakt (SEQ ID NO: 90), ETRA+74ErepS (SEQ ID NO: 91), ETRA+74Rakt (SEQ ID NO: 92), ETRA+74RrepS (SEQ ID NO: 93), ETRA+74Vakt (SEQ ID NO: 94), ETRA+74VrepS (SEQ ID NO: 95), ETRA+74AAakt (SEQ ID NO: 96), ETRA+74AArepS (SEQ ID NO: 97), ETRA+74ABakt (SEQ ID NO: 98), ETRA+74ABrepS (SEQ ID NO: 99), ETRA+74ACakt (SEQ ID NO:100), ETRA+74ACrepS (SEQ ID NO: 101), ETRA+74ADakt (SEQ ID NO: 102), ETRA+74ADrepS (SEQ ID NO: 103), ETRA+74AEakt (SEQ ID NO: 104), ETRA+74AErepS (SEQ ID NO: 105), ETRA+74AFakt (SEQ ID NO: 106), ETRA+74AFrepS (SEQ ID NO: 107), ETRA+74AGakt (SEQ ID NO: 108), ETRA+74AGrepS (SEQ ID NO: 109), ETRA+74AHakt (SEQ ID NO: 110), ETRA+74AHrepS (SEQ ID NO: 111), ETRA-37Bakt (SEQ ID NO: 112), ETRA-37BrepS (SEQ ID NO: 113), ETRA-37Dakt (SEQ ID NO: 114), ETRA-37DrepS (SEQ ID NO: 115), ETRA-50Aakt (SEQ ID NO: 116), ETRA-50ArepS (SEQ ID NO: 117), ETRA-50Bakt (SEQ ID NO: 118), ETRA-50BrepS (SEQ ID NO: 119), ETRA-50Cakt (SEQ ID NO: 120), ETRA-50CrepS (SEQ ID NO: 121), ETRA-50Dakt (SEQ ID NO: 122), ETRA-50DrepS (SEQ ID NO: 123), ETRA-50Eakt (SEQ ID NO: 124), ETRA-50ErepS (SEQ ID NO: 125), ETRA-50Fakt (SEQ ID NO: 126), ETRA-50FrepS (SEQ ID NO: 127), ETRA-50Gakt (SEQ ID NO: 128), ETRA-50GrepS (SEQ ID NO: 129), ETRA-50Hakt (SEQ ID NO: 130), ETRA-50HrepS (SEQ ID NO: 131), ETRA-50Iakt (SEQ ID NO: 132), ETRA-50IrepS (SEQ ID NO: 133), ETRA-50Jakt (SEQ ID NO: 134), ETRA-50JrepS (SEQ ID NO: 135), ETRA-50Kakt (SEQ ID NO: 136), ETRA-50KrepS (SEQ ID NO: 137), ETRA-50Lakt (SEQ ID NO: 138), ETRA-50LrepS (SEQ ID NO: 139), ETRA-50Makt (SEQ ID NO: 140), and ETRA-50MrepS (SEQ ID NO: 141). A cathepsin B non-sensitive artificial transcription factor is ETRA+74VrepSNPS (SEQ ID NO: 142). An inactive version of ETRA+74VrepS lacking all zinc coordinating cysteine residues for control purposes is ETRA+74Vmut_repS (SEQ ID NO: 143).

ETRB-specific hexameric zinc fingers are ETRB-1149H (SEQ ID NO: 144), ETRB-1149N (SEQ ID NO: 145), ETRB-487C (SEQ ID NO: 146), and ETRB-487E (SEQ ID NO: 147). Resulting ETRB-specific cathepsin B-sensitive VP64- (akt) or SID- (repS) containing transcription factors are ETRB-1149Hakt (SEQ ID NO: 148), ETRB-1149HrepS (SEQ ID NO: 149), ETRB-1149Nakt (SEQ ID NO: 150), ETRB-1149NrepS (SEQ ID NO: 151), ETRB-487Cakt (SEQ ID NO: 152), ETRB-487CrepS (SEQ ID NO: 153), ETRB-487Eakt (SEQ ID NO: 154), and ETRB-487ErepS (SEQ ID NO: 155).

TLR4-specific hexameric zinc fingers are TLR4-55B (SEQ ID NO: 156), TLR4-55E (SEQ ID NO: 157), TLR4-222A (SEQ ID NO: 158), TLR4-222B (SEQ ID NO: 159), TLR4-276B (SEQ ID NO: 160), and TLR4-276C (SEQ ID NO: 161). Resulting TLR4-specific cathepsin B-sensitive VP64- (akt) or SID- (repS) containing transcription factors are TLR4-55Bakt (SEQ ID NO: 162), TLR4-55BrepS (SEQ ID NO: 163), TLR4-55Eakt (SEQ ID NO: 164), TLR4-55ErepS (SEQ ID NO: 165), TLR4-222Aakt (SEQ ID NO: 166), TLR4-222ArepS (SEQ ID NO: 167), TLR4-222Bakt (SEQ ID NO: 168), TLR4-222BrepS (SEQ ID NO: 169), TLR4-276Bakt (SEQ ID NO: 170), TLR4-276BrepS (SEQ ID NO: 171), TLR4-276Cakt (SEQ ID NO: 172), and TLR4-276CrepS (SEQ ID NO: 173).

FCER1A-specific hexameric zinc fingers are IgER-147A (SEQ ID NO: 174), IgER-147G (SEQ ID NO: 175), IgER+17G (SEQ ID NO: 176), and IgER+17I (SEQ ID NO: 177). Resulting FCER1A-specific cathepsin B-sensitive VP64- (akt) or SID- (repS) containing transcription factors are IgER-147Aakt (SEQ ID NO: 178), IgER-147ArepS (SEQ ID NO: 179), IgER-147Gakt (SEQ ID NO: 180), IgER-147GrepS (SEQ ID NO: 181), IgER+17Gakt (SEQ ID NO: 182), IgER+17GrepS (SEQ ID NO: 183), IgER+171akt (SEQ ID NO: 184), and IgER+171repS (SEQ ID NO: 185). A cathepsin B non-sensitive artificial transcription factor is IgER-147ArepSNPS (SEQ ID NO: 186). An inactive version of IgER-147ArepS lacking all zinc coordinating cysteine residues for control purposes is IgER-147Amut_repS (SEQ ID NO: 187).

A TGFbR1-specific hexameric zinc finger protein is TGF-390A (SEQ ID NO: 188). Resulting TGFbR1-specific cathepsin B-sensitive VP64- (akt) or SID- (repS) containing transcription factors are TGF-390Aakt (SEQ ID NO: 189) and TGF-390repS (SEQ ID NO: 190).

In another embodiment, the artificial transcription factors targeting particular membrane-bound receptor gene promoters according to the invention comprise a zinc finger protein based on the zinc finger module composition of SEQ ID NO: 64 to 89, 144 to 147, 156 to 161, 174 to 177, and 188 wherein up to three, preferably one or two, individual zinc finger modules are exchanged against other zinc finger modules with alternative binding characteristic to modulate the binding of the artificial transcription factor to its target sequence, and/or wherein up to twelve, most preferably one or two individual amino acids are exchanged in order to minimize potential immunogenicity while retaining binding affinity to the intended target site.

In a particular embodiment, the artificial transcription factors targeting receptor gene promoters comprise a zinc finger protein based on the zinc finger module composition of SEQ ID NO: 64 to 89, 144 to 147, 156 to 161, 174 to 177, and 188, wherein optionally up to three, preferably one or two, individual zinc finger modules are exchanged against other zinc finger modules with alternative binding characteristic to modulate the binding of the artificial transcription factor to its target sequence and/or wherein optionally up to twelve, most preferably one or two individual amino acids are exchanged in order to minimize potential immunogenicity while retaining binding affinity to the intended target site, and wherein the transcription modulating domain is VP16, VP64, CJ7, p65-TA1, SAD, NF-1, AP-2, SP1-A, SP1-B, Oct-1, Oct-2, Oct2-5x, MTF-1, BTEB-2, LKLF, N-KRAB, C-KRAB, SID or ERD. More particularly the invention relates to such artificial transcription factors wherein the endosome-specific site is a cathepsin B cleavage site, and to such artificial transcription factors wherein the endosome-specific site is a cathespsin B cleavage site altered to minimize potential immunogenicity or cleavage specificity or efficiency.

### Transducible Artificial Transcription Factors Targeting Nuclear Receptor Promoter

Specific hexameric zinc finger proteins targeting specific target sites inside nuclear receptor promoters are composed of the Barbas zinc finger module set (Gonzalez B., 2010, Nat Protoc 5, 791-810) using the ZiFit software v3.3 (Sander J. D., Nucleic Acids Research 35, 599-605), or are selected using improved yeast one hybrid screening. To generate activating, cathepsin B-sensitive, transducible artificial transcription factors targeting the glucocorticoid receptor, hexameric zinc finger proteins proteins GR_ZFP1 (SEQ ID NO: 191), GR_ZFP2 (SEQ ID NO: 192), and GR_ZFP_{—}3 (SEQ ID NO: 193) are fused to the protein transduction domain TAT as well as the transcription activating domain VP64 yielding artificial transcription factors GR1akt (SEQ ID NO: 194), GR2akt (SEQ ID NO: 195) and GR3akt (SEQ ID NO: 196). To generate transducible cathepsin B-sensitive artificial transcription factors with negative regulatory activity, hexameric zinc finger proteins were fused to the protein transduction domain TAT as well as the transcription repressing domain SID yielding artificial transcription factors GR1rep (SEQ ID NO: 197), GR2rep (SEQ ID NO: 198) and GR3rep (SEQ ID NO: 199).

AR-specific hexameric zinc finger proteins are AR_ZFP1 (SEQ ID NO: 200), AR_ZFP2 (SEQ ID NO: 201), AR_ZFP3 (SEQ ID NO: 202), AR-236A (SEQ ID NO: 203), AR-236B (SEQ ID NO: 204), and AR-236C (SEQ ID NO: 205). Resulting AR-specific cathepsin B-sensitive VP64- (akt) or SID- (repS) containing artificial transcription factors are AR1akt (SEQ ID NO: 206), AR1repS (SEQ ID NO: 207), AR2akt (SEQ ID NO: 208), AR2repS (SEQ ID NO: 209), AR3akt (SEQ ID NO: 210), AR3repS (SEQ ID NO: 211), AR-236Aakt (SEQ ID NO: 212), AR-236ArepS (SEQ ID NO: 213), AR-236Bakt (SEQ ID NO: 214), AR-236BrepS (SEQ ID NO: 215), AR-236Cakt (SEQ ID NO: 216), and AR-236CrepS (SEQ ID NO: 217).

To generate activating transducible, cathepsin B-sensitive, artificial transcription factors targeting the estrogen receptor, hexameric zinc finger proteins ER_ZFP1 (SEQ ID NO: 218), ER_ZFP2 (SEQ ID NO: 219), and ER_ZFP_{—}3 (SEQ ID NO: 220) are fused to the protein transduction domain TAT as well as the transcription activating domain VP64 yielding artificial transcription factors ER1akt (SEQ ID NO: 221), ER2akt (SEQ ID NO: 222) and ER3akt (SEQ ID NO: 223). To generate transducible, cathepsin B-sensitive, artificial transcription factors with negative-regulatory activity, hexameric zinc finger proteins are fused to the protein transduction domain TAT as well as the transcription repressing domain SID yielding artificial transcription factors ER1rep (SEQ ID NO: 224), ER2rep (SEQ ID NO: 225) and ER3rep (SEQ ID NO: 226)

Considered are also artificial transcription factors of the invention containing pentameric, hexameric, heptameric or octameric zinc finger proteins, wherein individual zinc finger modules are exchanged to improve binding affinity towards target sites of the respective nuclear receptor promoter gene or to alter the immunological profile of the zinc finger protein for improved tolerability.

In another embodiment, the artificial transcription factors targeting particular nuclear receptor gene promoters according to the invention comprise a zinc finger protein based on the zinc finger module composition of SEQ ID NO: 191 to 193, 200 to 205, 218 to 220, wherein up to three, preferably one or two individual zinc finger modules are exchanged against other zinc finger modules with alternative binding characteristic to modulate the binding of the artificial transcription factor to its target sequence, and/or wherein up to twelve, for example twelve, eleven, ten or nine, in particular eight, seven, six or five, preferably four or three, most preferably one or two individual amino acids are exchanged in order to minimize potential immunogenicity while retaining binding affinity to the intended target site.

In a particular embodiment, the artificial transcription factors targeting nuclear receptor gene promoters comprise a zinc finger protein based on the zinc finger module composition of SEQ ID NO: 191 to 193, 200 to 205, 218 to 220, wherein optionally up to three, preferably one or two, individual zinc finger modules are exchanged against other zinc finger modules with alternative binding characteristic to modulate the binding of the artificial transcription factor to its target sequence, and/or wherein optionally up to twelve, most preferably one or two individual amino acids are exchanged in order to minimize potential immunogenicity while retaining binding affinity to the intended target site, and wherein the transcription modulating domain is VP16, VP64, CJ7, p65-TA1, SAD, NF-1, AP-2, SP1-A, SP1-B, Oct-1, Oct-2, Oct2-5x, MTF-1, BTEB-2, LKLF, N-KRAB, C-KRAB, SID or ERD. More particularly the invention relates to such artificial transcription factors, wherein the endosome-specific site is a cathepsin B cleavage site, and to such artificial transcription factors, wherein the endosome-specific site is a cathespsin B cleavage site altered to minimize potential immunogenicity or cleavage specificity or efficiency.

### Transducible Artificial Transcription Factors Targeting Haploinsufficient Gene Promoters

Specific hexameric zinc finger proteins are composed of the Barbas zinc finger module set (Gonzalez B., 2010, Nat Protoc 5, 791-810), using the ZiFit software v3.3 (Sander J. D., Nucleic Acids Research 35, 599-605) or are selected using improved yeast one hybrid screening.

OPA1-specific hexameric zinc finger proteins are OPA1_ZFP1 (SEQ ID NO: 227), OPA1_ZFP2 (SEQ ID NO: 228), OPA1-916B (SEQ ID NO: 229), OPA1-916C (SEQ ID NO: 230), OPA1-916D (SEQ ID NO: 231), OPA1-916E (SEQ ID NO: 232), OPA1-18B (SEQ ID NO: 233), OPA1-18C (SEQ ID NO: 234), OPA1-18D (SEQ ID NO: 235), OPA1-18E (SEQ ID NO: 236), OPA1-165A (SEQ ID NO: 237), OPA1-165B (SEQ ID NO: 238), OPA1-165C (SEQ ID NO: 239), OPA1-165D (SEQ ID NO: 240), OPA1-165E (SEQ ID NO: 241), OPA1-165F (SEQ ID NO: 242), OPA1-165G (SEQ ID NO: 243), and OPA1-165H (SEQ ID NO: 244). Corresponding OPA1-specific cathepsin B-sensitive artificial VP64-containing transcription factors are OPA_akt1 (SEQ ID NO: 245), OPA_akt2 (SEQ ID NO: 246), OPA1-916Bakt (SEQ ID NO: 247), OPA1-916Cakt (SEQ ID NO: 248), OPA1-916Dakt (SEQ ID NO: 249), OPA1-916Eakt (SEQ ID NO: 250), OPA1-18Bakt (SEQ ID NO: 251), OPA1-18Cakt (SEQ ID NO: 252), OPA1-18Dakt (SEQ ID NO: 253), OPA1-18Eakt (SEQ ID NO: 254), OPA1-165Aakt (SEQ ID NO: 255), OPA1-165Bakt (SEQ ID NO: 256), OPA1-165Cakt (SEQ ID NO: 257), OPA1-165Dakt (SEQ ID NO: 258), OPA1-165Eakt (SEQ ID NO: 259), OPA1-165Fakt (SEQ ID NO: 260), OPA1-165Gakt (SEQ ID NO: 261), and OPA1-165Hakt (SEQ ID NO: 262).

Considered are also artificial transcription factors of the invention containing pentameric, hexameric, heptameric or octameric zinc finger proteins, wherein individual zinc finger modules are exchanged to improve binding affinity towards target sites of the respective haploinsufficient promoter gene or to alter the immunological profile of the zinc finger protein for improved tolerability.

In another embodiment, the artificial transcription factors targeting haploinsufficient gene promoters according to the invention comprise a zinc finger protein based on the zinc finger module composition of SEQ ID NO: 227 and 244, wherein up to three, preferably one or two, individual zinc finger modules are exchanged against other zinc finger modules with alternative binding characteristic to modulate the binding of the artificial transcription factor to its target sequence, and/or, wherein up to up to twelve, most preferably one or two individual amino acids are exchanged in order to minimize potential immunogenicity while retaining binding affinity to the intended target site.

In a particular embodiment, the artificial transcription factors targeting haploinsufficient gene promoters comprise a zinc finger protein based on the zinc finger module composition of SEQ ID NO: 227 and 244, wherein optionally up to three, preferably one or two, individual zinc finger modules are exchanged against other zinc finger modules with alternative binding characteristic to modulate the binding of the artificial transcription factor to its target sequence and/or wherein optionally up to twelve, most preferably one or two individual amino acids are exchanged in order to minimize potential immunogenicity while retaining binding affinity to the intended target site, and wherein the transcription modulating domain is VP16, VP64, CJ7, p65-TA1, SAD, NF-1, AP-2, SP1-A, SP1-B, Oct-1, Oct-2, Oct2-5x, MTF-1, BTEB-2, LKLF, N-KRAB, C-KRAB, SID or ERD. More particularly the invention relates to such artificial transcription factors, wherein the endosome-specific site is a cathepsin B cleavage site, and to such artificial transcription factors, wherein the endosome-specific site is a cathespsin B cleavage site altered to minimize potential immunogenicity or cleavage specificity or efficiency.

### Activity of Artificial Transcription Factors in Regulating Receptor Promoter Activity

To assess the potential of artificial transcription factors to influence transcription driven by the receptor promoter, a luciferase reporter assay was employed (FIG. 2). To this end, HeLa cells capable of driving expression from the ETRA promoter were co-transfected with an artificial transcription factor expression plasmid together with a dual-reporter plasmid. The dual-reporter plasmid contained the secreted Gaussia luciferase gene under the control of the ETRA promoter together with the gene for secreted alkaline phosphatase (SEAP) under control of the constitutive CMV promoter based on the NEG-PG04 and EF1a-PG04 plasmids (GeneCopoeia, Rockville, Md.). This co-transfection was done in a 3:1 artificial transcription factor expression plasmid:reporter plasmid ratio to ensure the presence of artificial transcription factor expression in cells transfected with the reporter plasmid, and Gaussia luciferase, and SEAP activity was measured according to manufacturer's recommendation (Gaussia Luciferase Glow Assay Kit, Pierce; SEAP Reporter Gene Assay Chemiluminescence, Roche). Luciferase values were normalized to SEAP activity and compared to control cells expressing yellow fluorescent protein (YFP) set to 100%. By measuring the ratio between luciferase and SEAP activity in the supernatant of transfected cells, normalization of receptor promoter-driven luciferase expression to SEAP expression only in cells transfected with artificial transcription factor plasmid was possible. This approach proved useful to account and normalize for differences in transfection efficiency between different experiments and allowed for quantification of artificial transcription factor mediated regulation of a given receptor promoter. The luciferase expression studies were performed at least three times in triplicates, averaged, compared to control transfected cells, expressed as relative luciferase activity (RLuA) in % of control and plotted with error bars depicting SEM. Expression of AO74V suppressed ETRA promoter-driven expression to 3.2% compared to control cells.

### Accessibility of the ETRA_TS+74 Binding Site for the ETRA-Specific Artificial Transcription Factor AO74V in the Endogenous Gene.

In order to exert regulatory activity, artificial transcription factors need to be able to bind to their target site in the context of the endogenous genomic region. To analyze whether artificial transcription factors containing the ETRA+74V zinc finger protein (SEQ ID NO: 80) are capable of binding to their target site in the ETRA gene (ETRA_TS+74, SEQ ID NO: 41), stable cell lines containing an expression construct for AO74V under control of a tetracycline-inducible promoter were generated. Induction of these cells with tetracycline for 24 hours caused the production of AO74V protein (SEQ ID NO: 263), while no AO74V was produced in the absence of tetracycline. As shown in FIG. 3A, expression of AO74V caused the almost complete loss of ETRA mRNA in HEK 293 FlpIn cells compared to un-induced cells or cells expressing an inactive variant of AO74V lacking DNA binding capability or an empty vector control. While cells in FIG. 3A contained the expression constructs integrated into the FlpIn site, HEK 293 FlpIn TRex cells shown in FIG. 3B contained the tetracycline-inducible expression constructs in the AAVS1 safe harbor locus. Also in these cells, expression of AO74V but not of inactive AO74V caused the almost complete suppression of ETRA expression. Again using HeLa cells stably containing tetracycline-inducible expression constructs for AO74V, inactive AO74V or empty vector control in the AAVS1 locus (FIG. 3C), induction of AO74V but not of inactive AO74V using tetracycline did result in the strong suppression of ETRA expression. Taken together, the ETRA_TS+74 target site in the endogenous ETRA promoter is accessible for artificial transcription factors, and upon binding to this target site artificial transcription factors containing the SID negative-regulatory domain are in a position permissive for the suppression of ETRA expression.

### Assessment of ET-1 Dependent Calcium Signaling Following Expression of ETRA-Specific Artificial Transcription Factor

The ETRA agonist ET-1 stimulates calcium flux in HEK 293 FlpIn TRex cells. Therefore, suppression of ETRA expression is expected to suppress such changes in the intracellular calcium concentration following stimulation with ET-1. HEK 293 FlpIn TRex expressing AO74V (SEQ ID NO: 263 were induced with tetracycline for 48 hours and were treated with 0, 100, 1000 nM ET-1 and calcium flux was measured using a calcium-sensitive fluorescent dye (Calcium 5 Assay Kit, Molecular Devices) using an automated fluorescence plate reader (FlexStation 3, Molecular Devices). Cells not induced with tetracycline served as control. As shown in FIG. 4A, ET-1 is able to induce a concentration-dependent increase in intracellular calcium concentrations in cells not expressing the artificial transcription factor, while cells expressing the ETRA-specific artificial transcription factor no longer respond to ET-1 stimulation (FIG. 4B). These data are consistent with a loss of ETRA-dependent signaling due to a lack of ETRA protein following expression of this artificial transcription factor.

### Assessment of ET-1 Dependent Contraction of Human Uterine Smooth Muscle Cells Following Application of ETRA-Specific Artificial Transcription Factor

Smooth muscle cells (SMCs) express ETRA and are capable of contraction following exposure to ET-1. To measure the effectiveness of anti-ETRA promoter artificial transcription factor ETRA+74VrepSNPS (SEQ ID NO: 142), human uterine smooth muscle cells (hUtSMCs) were used as model system. To this end, hUtSMCs were embedded into 3-dimensional collagen lattices and treated for three days with 1 μM ETRA+74VrepSNPS or buffer control before exposure to 0 or 100 nM ET-1. The protein or buffer treatment was repeated every 24 hours. Following detachment of the lattices from their support and addition of ET-1, contraction of lattices was observed. As shown in FIG. 5, control lattices exposed to ET-1 contract to about 78% compared to lattices not treated with ET-1. In contrast, ETRA+74VrepSNPS treated lattices did not significantly contract in the presence of ET-1 when compared to control lattices not treated with ET-1. This is consistent with a complete block of ET-1 induced contraction of hUtSMCs following treatment with ETRA+74VrepNPS. The data shown in FIG. 4 represents the average lattice area 9 hours after ET-1 addition of three independent experiments done in sextuplicates. Statistical analysis using the SPSS software package employing a general linear univariate model revealed high significance (** represent p<0.001) for the blocking action of ETRA+74VrepNPS.

### Increased Nuclear Localization of Cathepsin B-Sensitive ETRA Specific Artificial Transcription Factor

To assess whether the addition of an endosome-specific protease cleavage site indeed improves the subcellular targeting of artificial transcription factors of the invention, HeLa cells were transduced with ETRA-specific artificial transcription factor protein

ETRA+74VrepSNPS(a variant of ETRA+74VrepS lacking the cathepsin B site containing the SID negative regulatory domain) or cathepsin B sensitive ETRA+74VrepS protein, and nuclear localization was analyzed by fluorescence microscopy followed by image analysis. As shown in FIG. 6, the incorporation of a cathepsin B cleavage site increased the mean concentration of artificial transcription factor in the nucleus 4.7 fold. Cells transduced with the cathepsin B-sensitive ETRA+74VrepS also showed a more uniform uptake of artificial transcription factor into the nucleus with 75% of cells reaching up to 47.5% of the maximal concentration, while 75% of cells transduced with the cathepsin B-insensitive ETRA+74VrepSNPS are below 10.4% of the maximal concentration. These data are consistent with a cathepsin B-dependent cleavage of ETRA+74VrepS in the endosomal compartment resulting in the separation of the TAT protein transduction domain from the remainder of the artificial transcription factor. This allows for the efficient escape from the endosomal compartment of the artificial transcription factor part of ETRA+74VrepS once stochastic vesicle rupture occurred.

### Inclusion of a Cathepsin B Site Increases Activity of Transducible Artificial Transcription Factor in a Luciferase Reporter Assay

As shown above, the cathepsin B-sensitive ETRA-specific artificial transcription factor ETRA+74VrepS localizes more efficiently to the nuclear compartment following protein transduction compared to the cathepsin B-insensitive ETRA+74VrepSNPS. To assess whether this improved nuclear localization translates into increased activity in terms of transcriptional regulation, a luciferase reporter assay was employed. To this end, HEK 293 cells containing a reporter construct consisting of Gaussia luciferase under the control of a hybrid CMV/ETRA_TS+74 promoter and secreted alkaline phosphatase were treated for 2 hours with 1 μM ETRA+74VrepS, ETRA+74VrepSNPS or an inactive version of ETRA+74VrepS as control, and luciferase and secreted alkaline phosphatase activity was measured 24 hours after treatment. As shown in FIG. 7, luciferase activity is decreased to 57.9+/−5.8% following treatment with ETRA+74VrepSNPS compared to control, while treatment with ETRA+74VrepS reduces luciferase activity to 87.2+/−8.2%. These data support the notion that increased nuclear localization of an artificial transcription factor due to increased cathepsin B-mediated endosomal escape translates into increased activity in terms of transcriptional regulation.

Similar results were obtained when comparing cathepsin B-sensitive artificial transcription factors targeting the TLR4, AR, or FcER1A promoter with the respective cathepsin B-insensitive variants using reporter cell lines containing luciferase under hybrid CMV promoters responsive to the respective artificial transcription factor. As shown in FIG. 8, treatment of suitable reporter cells with the cathepsin B-sensitive TLR4-222BrepS artificial transcription factor reduced relative luciferase activity to 61.3+/−6.9% compared to control treated cells, while treatment with the cathepsin B-insensitive TLR4-222BrepSNSP did not result in a suppression of luciferase activity. Similarly, treatment of suitable reporter cells with the cathepsin B-cleavable AR-236ArepS resulted in the reduction of relative luciferase activity to 52+/−11% compared to control, while treatment with AR-236ArepSNPS reduced luciferase activity only to 85+/−11% of control treated cells. Furthermore, treatment of suitable reporter cells with the cathepsin B-sensitive IgER-147ArepS caused a reduction of relative luciferase activity to 52.7+/−12.9% compared to control treated cells, while the corresponding cathepsin B-insensitive IgER-147ArepSNPS did not cause a reduction in luciferase activity compared to control cells. Taken together, the inclusion of a cathepsin B cleavage site into transducible artificial transcription factors greatly enhanced not only their correct nuclear localization but also their activity in terms of transcriptional regulation. Thus, separating the protein transduction domain with its high affinity for cellular membrane from the artificial transcription factor through the action of an endosomal protease allows for efficient exit of active artificial transcription factor following rupture of endosomal vesicles.

### An ETRA-Specific Cathepsin B-Sensitive Artificial Transcription Factor Shows Activity in Human Tissue

Suppression of ETRA expression through the action of an ETRA-specific artificial transcription factor is expected to interfere with endothelin-dependent, ETRA-mediated cellular signaling. Endothelin is the strongest known vasoconstrictor known, thus, downregulation of the endothelin receptor ETRA is predicted to block endothelin-dependent vasoconstriction. To assess whether ETRA+74VrepS is capable of influencing ETRA levels and thus block endothelin-dependent vasoconstriction, vasoconstriction of ex vivo human vessels treated with ETRA+74VrepS was measured. To this end, isolated human coronary artery vessel rings were incubated for three days in the presence of 1 μM ETRA+74VrepS. Incubation with vehicle served as control. To assess vessel contractibility, vessel rings were mounted into a wire myograph and vessel response to the ETRA-independent vasoconstrictor U46619 as well as increasing concentrations of endothelin was measured. As shown in FIG. 11, treatment with ETRA+74VrepS did reduce relative endothelin-dependent vessel contraction compared to vehicle treated control vessels. These data are consistent with the downregulation of ETRA gene expression resulting in the reduction of ETRA protein levels and a subsequent decrease in endothelin-dependent vasoconstriction in human coronary arteries through die action of ETRA+74VrepS.

The FCER1A-specific, cathepsin B-sensitive artificial transcription factor IgER-147ArepS shows activity in a humanized mouse model of anaphylactic shock.

Crosslinking IgE receptors on mast cells or basophiles through the binding of a multivalent antigen causes the release of allergic mediators such as histamine and others from these cells. In case of system activation of the process, e.g. following systemic exposure to an allergen, systemic release of histamine occurs, leading together with the other allergic mediators to anaphylactic shock. The anaphylactic shock is characterized by edema, a drop in blood pressure and hypothermia. To model anaphylaxis in animals, the following strategy is applied: Animals are sensitized through injection of a specific IgE antibody, for example raised against dinitrophenyl (DNP). The injected specific IgE now binds to IgE receptors on mast cells and basophiles priming these cells for the release of allergic mediators. To now activate these cells, DNP coupled to BSA in a high molar ratio is injected and leads upon binding to the crosslinking of IgE receptors through the bound specific anti-DNP IgE.

As IgER-147ArepS is targeting the FCER1A-promoter leading to a loss of IgE receptor, pretreatment with this artificial transcription factor will interfere with the induction of anaphylaxis, as the release of allergic mediators critically depends on the IgE receptor. To assess the activity of IgER-147ArepS in an in vivo model, a humanized mouse model (hNSG) was chosen, since this artificial transcription factor is selected against the human FCER1A promoter and is not expected to suppress expression from the murine FCER1A promoter. The hNSG model is based on the severely immune-compromised NSG mouse, which was implanted with human CD34+ stem cells capable of generating a quasi human immune system in a mouse. As shown in FIG. 12, hNSG mice pretreated twice with IgER-147ArepS artificial transcription factors at five days and two days prior to induction of anaphylaxis using anti-DNP IgE/DNP-BSA were less susceptible to anaphylactic shock compared to control animals. While anaphylaxis in vehicle treated control animals resulted in rapid death of the animal ten minutes post induction, IgER-147ArepS treated animals survived anaphylaxis for sixty minutes. These data are a clear indicator of suppression of anaphylaxis through diminished IgER activity following treatment with IgER-147ArepS.

### Attachment of a Polyethylene Glycol Residue

The covalent attachment of a polyethylene glycol residue (PEGylation) to an artificial transcription factor of the invention is considered to increase solubility of the artificial transcription factor, to decrease its renal clearance, and control its immunogenicity. Considered are amine as well as thiol reactive polyethylene glycols ranging in size from 1 to 40 Kilodalton. Using thiol reactive polyethylene glycols, site-specific PEGylation of the artificial transcription factors is achieved. The only essential thiol group containing amino acids in the artificial transcription factors of the invention are the cysteine residues located in the zinc finger modules essential for zinc coordination. These thiol groups are not accessible for PEGylation due their zinc coordination, thus, inclusion of one or several cysteine residues into the artificial transcription factors of the invention provides free thiol groups for PEGylation using thiol-specific polyethylene glycol reagents.

### Pharmaceutical Compositions

The present invention relates also to pharmaceutical compositions comprising an artificial transcription factor as defined above. Pharmaceutical compositions considered are compositions for parenteral systemic administration, in particular intravenous administration, compositions for inhalation, and compositions for local administration, in particular ophthalmic-topical administration, e.g. as eye drops, eye gels or sprays, or intravitreal, subconjunctival, parabulbar or retrobulbar administration, to warm-blooded animals, especially humans. Particularly preferred are eye drops and eye gels and compositions for intravitreal, subconjunctival, parabulbar or retrobulbar administration. The compositions comprise the active ingredient alone or, preferably, together with a pharmaceutically acceptable carrier. Further considered are slow-release formulations. The dosage of the active ingredient depends upon the disease to be treated and upon the species, its age, weight, and individual condition, the individual pharmacokinetic data, and the mode of administration.

Further considered are pharmaceutical compositions useful for oral delivery, in particular compositions comprising suitably encapsulated active ingredient, or otherwise protected against degradation in the gut. For example, such pharmaceutical compositions may contain a membrane permeability enhancing agent, a protease enzyme inhibitor, and be enveloped by an enteric coating.

The pharmaceutical compositions comprise from approximately 1% to approximately 95% active ingredient. Unit dose forms are, for example, ampoules, vials, inhalers, eye drops, eye gels and the like.

The pharmaceutical compositions of the present invention are prepared in a manner known per se, for example by means of conventional mixing, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80™ (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

For parenteral administration, aqueous solutions of the active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Compositions for inhalation can be administered in aerosol form, as sprays, mist or in form of drops. Aerosols are prepared from solutions or suspensions that can be delivered with a metered-dose inhaler or nebulizer, i.e. a device that delivers a specific amount of medication to the airways or lungs using a suitable propellant, e.g. dichlorodifluoro-methane, trichlorofluoromethane, dichlorotetrafluoroethane, carbon dioxide or other suitable gas, in the form of a short burst of aerosolized medicine that is inhaled by the patient. It is also possible to provide powder sprays for inhalation with a suitable powder base such as lactose or starch.

Eye drops are preferably isotonic aqueous solutions of the active ingredient comprising suitable agents to render the composition isotonic with lacrimal fluid (295-305 mOsm/l). Agents considered are sodium chloride, citric acid, glycerol, sorbitol, mannitol, ethylene glycol, propylene glycol, dextrose, and the like. Furthermore the composition comprise buffering agents, for example phosphate buffer, phosphate-citrate buffer, or Tris buffer (tris(hydroxymethyl)-aminomethane) in order to maintain the pH between 5 and 8, preferably 7.0 to 7.4. The compositions may further contain antimicrobial preservatives, for example parabens, quaternary ammonium salts, such as benzalkonium chloride, polyhexamethylene biguanidine (PHMB) and the like. The eye drops may further contain xanthan gum to produce gel-like eye drops, and/or other viscosity enhancing agents, such as hyaluronic acid, methylcellulose, polyvinylalcohol, or polyvinylpyrrolidone.

### Use of Artificial Transcription Factors in a Method of Treatment

Furthermore the invention relates an artificial transcription factors directed to the endothelin receptor A promoter as described above for use in influencing the cellular response to endothelin, for lowering or increasing endothelin receptor levels, and for use in the treatment of diseases modulated by endothelin, in particular for use in the treatment of such eye diseases. Likewise the invention relates to a method of treating a disease modulated by endothelin comprising administering a therapeutically effective amount of an artificial transcription factor directed to the endothelin receptor A promoter to a patient in need thereof.

Diseases modulated by endothelin are, for example, cardiovascular diseases such as essential hypertension, pulmonary hypertension, chronic heart failure but also chronic renal failure. In addition, renal protection before, during and after radioopaque material application is achieved by blunting the endothelin response. In addition, multiple sclerosis is negatively impacted by the endothelin system.

Further diseases modulated by endothelin are diabetic kidney disease or eye diseases such as glaucomatous neurodegeneration, vascular dysregulation in ocular blood circulation, retinal vein occlusion, retinal artery occlusion, macular edema, age related macula degeneration, optic neuropathy, central serous chorioretinopathy, retinitis pigmentosa, Susac syndrome, and Leber's hereditary optic neuropathy.

Likewise the invention relates to a method of treating a disease modulated by endothelin comprising administering a therapeutically effective amount of an artificial transcription factor of the invention to a patient in need thereof. In particular the invention relates to a method of treating glaucomatous neurodegeneration, vascular dysregulation in ocular blood circulation, in particular to a method of treating retinal vein occlusion, retinal artery occlusion, macular edema, optic neuropathy, central serous chorioretinopathy, retinitis pigmentosa, and Leber's hereditary optic neuropathy, comprising administering an effective amount of an artificial transcription factor of the invention to a patient in need thereof. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore the invention relates to an artificial transcription factor directed to the endothelin receptor B promoter as described above for use in influencing the cellular response to endothelin, for lowering or increasing endothelin receptor B levels, and for use in the treatment of diseases modulated by endothelin, in particular for use in the treatment of such eye diseases. Likewise the invention relates to a method of treating a disease modulated by endothelin comprising administering a therapeutically effective amount of an artificial transcription factor directed to the endothelin receptor B promoter to a patient in need thereof.

Diseases modulated by ET-1-dependent, ETRB-mediated artificial transcription factors are certain cancers, neurodegeneration and inflammation-related disorders.

Furthermore the invention relates to an artificial transcription factor directed to the TLR4 promoter as described above for use in influencing the cellular response to LPS, for lowering or increasing TLR4 levels, and for use in the treatment of diseases modulated by LPS, in particular for use in the treatment of such eye diseases. Likewise the invention relates to a method of treating a disease modulated by LPS comprising administering a therapeutically effective amount of an artificial transcription factor directed to the TLR4 promoter to a patient in need thereof. Diseases modulated by LPS are rheumatoid arthritis, artherosclerosis, psoriasis, Crohn's disease, uveitis, contact lens associated keratitis, corneal inflammation, resistance of cancers to chemotherapy and the like.

Furthermore the invention relates to an artificial transcription factor directed to the FCER1A promoter as described above for use in influencing the cellular response to IgE or IgE-antigen complexes, for lowering or increasing FCER1 levels, and for use in the treatment of diseases modulated by IgE or IgE-antigen complexes, in particular for use in the treatment of such eye diseases.

Likewise the invention relates to a method of treating a disease modulated by IgE or IgE-antigen complexes comprising administering a therapeutically effective amount of an artificial transcription factor directed to the FCER1A promoter to a patient in need thereof. Diseases modulated by IgE or IgE-antigen complexes are in general type I reactions according to the Coombs and Gell classification (Gell P. and Coombs R. (eds), 1968, Clinical Aspects of Immunology, Blackwell Scientific, Oxford). Such reactions include allergic rhinitis, asthma, atopic dermatitis, pollen allergy, food allergy, hay fever, respiratory allergy, pet allergy, dust allergy, dust mite allergy, allergic uriticaria, allergic alveolitis, allergic aspergillosis, allergic bronchitis, allergic blepharitis, allergic contact dermatitis, allergic conjunctivitis, allergic fungal sinusitis, allergic gastroenteritis, allergic interstitial nephritis, allergic keratitis, allergic laryngitis, allergic purpura, allergic urethritis, allergic vasculitis, eczema, anaphylaxis and the like.

Furthermore, the invention relates an artificial transcription factor assembled as to target the promoter region of a nuclear receptor as described above for use in influencing the cellular response to the nuclear receptor ligand, for lowering or increasing the levels of the nuclear receptor, and for use in the treatment of diseases modulated by such nuclear receptors. Likewise, the invention relates to a method of treating diseases modulated by a nuclear receptor ligand comprising administering a therapeutically effective amount of an artificial transcription factor directed to a nuclear receptor promoter to a patient in need thereof.

Diseases modulated by ligands of nuclear receptors are, for example, adrenal insufficiency, adrenocortical insufficiency, alcoholism, Alzheimer's disease, androgen insensitivity syndrome, anorexia nervosa, aortic aneurysm, aortic valve sclerosis, arthritis, asthma, atherosclerosis, attention deficit hyperactivity disorder, autism, azoospermia, biliary primary cirrhosis, bipolar disorder, bladder cancer, bone cancer, breast cancer, cardiovascular disease, cardiovascular myocardial infarction, celiac disease, cholestasis, chronic kidney failure and metabolic syndrome, cirrhosis, cleft palate, colorectal cancer, congenital adrenal hypoplasia, coronary heart disease, cryptorchidism, deep vein thrombosis, dementia, depression, diabetic retinopathy, endometriosis, endometrial cancer, enhanced S-cone syndrome, essential hypertension, familial partial lipodystrophy, glioblastoma, glucocorticoid resistance, Graves' Disease, high serum lipid levels, hyperapobetalipoproteinemia, hyperlipidemia, hypertension, hypertriglyceridemia, hypogonadotropic hypogonadism, hypospadias, infertility, inflammatory bowel disease, insulin resistance, ischemic heart disease, liver steatosis, lung cancer, lupus erythematosus, major depressive disorder, male breast cancer, metabolic plasma lipid levels, metabolic syndrome, migraine, mulitple sclerosis, myocardial infarct, nephrotic syndrome, non-Hodgkin's lymphoma, obesity, osteoarthritis, osteopenia, osteoporosis, ovarian cancer, Parkinson's disease, preeclampsia, progesterone resistance, prostate cancer, pseudohypoaldosteronism, psoriasis, psychiatric schizophrenia, psychosis, retinitis pigmentosa-37, schizophrenia, sclerosing cholangitis, sex reversal, skin cancer, spinal and bulbar atrophy of Kennedy, susceptibility to myocardial infarction, susceptibility to psoriasis, testicular cancer, type I diabetes, type II diabetes, uterine cancer and vertigo.

Likewise, the invention relates to a method of treating a disease modulated by ligands of nuclear receptors comprising administering a therapeutically effective amount of an artificial transcription factor of the invention to a patient in need thereof. In particular, the invention relates to a method of treating adrenal insufficiency, adrenocortical insufficiency, alcoholism, Alzheimer's disease, androgen insensitivity syndrome, anorexia nervosa, aortic aneurysm, aortic valve sclerosis, arthritis, asthma, atherosclerosis, attention deficit hyperactivity disorder, autism, azoospermia, biliary primary cirrhosis, bipolar disorder, bladder cancer, bone cancer, breast cancer, cardiovascular disease, cardiovascular myocardial infarction, celiac disease, cholestasis, chronic kidney failure and metabolic syndrome, cirrhosis, cleft palate, colorectal cancer, congenital adrenal hypoplasia, coronary heart disease, cryptorchidism, deep vein thrombosis, dementia, depression, diabetic retinopathy, endometriosis, endometrial cancer, enhanced S-cone syndrome, essential hypertension, familial partial lipodystrophy, glioblastoma, glucocorticoid resistance, Graves' Disease, high serum lipid levels, hyperapobetalipoproteinemia, hyperlipidemia, hypertension, hypertriglyceridemia, hypogonadotropic hypogonadism, hypospadias, infertility, inflammatory bowel disease, insulin resistance, ischemic heart disease, liver steatosis, lung cancer, lupus erythematosus, major depressive disorder, male breast cancer, metabolic plasma lipid levels, metabolic syndrome, migraine, mulitple sclerosis, myocardial infarct, nephrotic syndrome, non-Hodgkin's lymphoma, obesity, osteoarthritis, osteopenia, osteoporosis, ovarian cancer, Parkinson's disease, preeclampsia, progesterone resistance, prostate cancer, pseudohypoaldosteronism, psoriasis, psychiatric schizophrenia, psychosis, retinitis pigmentosa-37, schizophrenia, sclerosing cholangitis, sex reversal, skin cancer, spinal and bulbar atrophy of Kennedy, susceptibility to myocardial infarction, susceptibility to psoriasis, testicular cancer, type I diabetes, type II diabetes, uterine cancer and vertigo, comprising administering an effective amount of an artificial transcription factor of the invention to a patient in need thereof. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore, the invention relates an artificial transcription factor directed to the glucocorticoid receptor as described above for use in influencing the cellular response to ligands of the glucocorticoid receptor, for lowering or increasing glucocorticoid receptor levels, and for the use in the treatment of diseases modulated by ligands of the glucocorticoid receptor.

Likewise the invention relates to a method of treating a disease modulated by ligands of the glucocorticoid receptor comprising administering a therapeutically effective amount of an artificial transcription factor of the invention to a patient in need thereof. Diseases considered are glucocorticoid resistance, type II diabetes, obesity, coronary atherosclerosis, coronary artery disease, asthma, celiac disease, lupus erythematosus, depression, stress and nephrotic syndrome. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore, the invention relates an artificial transcription factor directed to the androgen receptor as described above for use in influencing the cellular response to ligands of the androgen receptor, for lowering or increasing androgen receptor levels, and for the use in the treatment of diseases modulated by ligands of the androgen receptor.

Likewise the invention relates to a method of treating a disease modulated by ligands of the androgen receptor comprising administering a therapeutically effective amount of an artificial transcription factor of the invention to a patient in need thereof. Diseases considered are prostate cancer, male breast cancer, ovarian cancer, colorectal cancer, endometrial cancer, testicular cancer, coronary artery disease, type I diabetes, diabetic retinopathy, obesity, androgen insensitivity syndrome, osteoporosis, osteoarthritis, type II diabetes, Alzheimer's disease, migraine, attention deficit hyperactivity disorder, depression, schizophrenia, azoospermia, endometriosis, and spinal and bulbar atrophy of Kennedy. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore, the invention relates an artificial transcription factor directed to the estrogen receptor as described above for use in influencing the cellular response to ligands of the estrogen receptor, for lowering or increasing estrogen receptor levels, and for the use in the treatment of diseases modulated by ligands of the estrogen receptor.

Likewise the invention relates to a method of treating a disease modulated by ligands of the estrogen receptor comprising administering a therapeutically effective amount of an artificial transcription factor of the invention to a patient in need thereof. Diseases considered are bone cancer, breast cancer, colorectal cancer, endometrial cancer, prostate cancer uterine cancer, alcoholism, migraine, aortic aneurysm, susceptibility to myocardial infarction, aortic valve sclerosis, cardiovascular disease, coronary artery disease, hypertension, deep vein thrombosis, Graves' Disease, arthritis, mulitple sclerosis, cirrhosis, hepatitis B, chronic liver disease, cholestasis, hypospadias, obesity, osteoarthritis, osteopenia, osteoporosis, Alzheimer's disease, Parkinson's disease, migraine, vertigo), anorexia nervosa, attention deficit hyperactivity disorder, dementia, depression, psychosis, endometriosis and infertility. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore, the invention relates an artificial transcription factor assembled as to target the promoter region of a haploinsufficient gene as described above for use in restoring gene production to physiological levels in order to alleviate pathological phenotypes caused by insufficient gene production expression. Likewise, the invention relates to a method of treating diseases caused or modulated by haploinsufficiency comprising administering a therapeutically effective amount of an artificial transcription factor directed to a haploinsufficient gene promoter.

Diseases considered in the present invention are Leri-Weill dyschondrosteosis, frontotemporal lobar degeneration with TDP43 inclusions, Kleefstra syndrome, Digeorge syndrome, neurofibromatosis type I, Pitt-Hopkins syndrome, mandibulofacial dysostosis with microcephaly, Williams-Beuren syndrome, autosomal dominant Ehlers-Danlos syndrome type Iv, dopa-responsive dystonia due to sepiapterin reductase deficiency, oculocutaneous albinism type II, Smith-Magenis syndrome, hypoparathyroidism, sensorineural deafness and renal disease (Hdr), Stickler syndrome type I, Mowat-Wilson syndrome, syndromic Microphthalmia 3, Ehlers-Danlos syndrome type III, aniridia, pseudohypoparathyroidism type Ia, early infantile epileptic encephalopathy 4, skin fragility-woolly hair syndrome, Miller-Dieker lissencephaly syndrome, Wolf-Hirschhorn syndrome, trichorhinophalangeal syndrome type I, otodental dysplasia, otodental syndrome with coloboma, myotonic dystrophy 1, Treacher-Collins syndrome 1, familial acne inverse 1, Ehlers-Danlos syndrome type I, brachydactyly-mental retardation syndrome, velocardiofacial syndrome, Ulnar-Mammary syndrome, campomelic dysplasia, early infantile epileptic encephalopathy 5, Koolen-De Vries syndrome, holoprosencephaly 5, syndromic microphthalmia 6, Dravet syndrome, Glut1 deficiency syndrome 1, neurodegeneration with brain iron accumulation 3, autosomal recessive juvenile Parkinson disease 2, synpolydactyly 1, supravalvular aortic stenosis, dominant optic atrophy 1, Carney complex type 1, Pallister-Hall syndrome, Holt-Oram syndrome, alpha-thalassemia/mental retardation syndrome, seizures, benign familial neonatal 1, alagille syndrome 1, brachydactyly type C, familial platelet disorder with associated myeloid malignancy, pancreatic agenesis and congenital heart defects, telomere-related pulmonary fibrosis and/or bone marrow failure 1, mirror movements 2, speech-language disorder 1, autosomal dominant deafness 9, Kenny-Caffey syndrome type 1, ataxia-telangiectasia, parietal foramina, Feingold syndrome 1, nail-patella syndrome, autosomal dominant mental retardation 1, holoprosencephaly 3, congenital clubfoot with or without deficiency of long bones and/or mirror-image polydactyly, Sotos syndrome 1, Loeys-Dietz syndrome type 4, idiopathic basal ganglia calcification 3, trigonocephaly 2, centronuclear myopathy 3, cognitive impairment with or without cerebellar ataxia, familial partial lipodystrophy type 4, mononeuropathy of the median nerve, Waardenburg syndrome type 4c, Waardenburg syndrome type 4b, atypical hemolytic uremic syndrome 5, autosomal dominant spastic paraplegia 42, pseudohypoparathyroidism, autosomal dominant spastic paraplegia 31, autosomal dominant progressive external ophthalmoplegia with mitochondrial DNA deletions 4, spinocerebellar ataxia 27, Charcot-Marie-tooth disease type 2a2, autosomal dominant auditory neuropathy 1, synpolydactyly 2, limb-girdle muscular dystrophy type 1c, lissencephaly 1, spinocerebellar ataxia 15, Ehlers-Danlos-like syndrome, hereditary motor and sensory neuropathy typellc, hairy elbows with short stature facial dysmorphism and developmental delay, Axenfeld-Rieger syndrome type 3, familial infantile convulsions with paroxysmal choreoathetosis, acute myeloid leukemia, Charcot-Marie-tooth disease type 2d, congenital cataracts with sensorineural deafness, Down syndrome-like facial appearance with short stature and mental retardation, autosomal dominant deafness 5, hyperferritinemia with or without cataract, oblique facial clefting 1, autosomal dominant deafness 2a, early infantile epileptic encephalopathy 1, susceptibility to autism X-Linked 2, Usher syndrome type IIIa, thrombocytopenia-absent radius syndrome, autosomal recessive Robinow syndrome, alveolar capillary dysplasia with misalignment of pulmonary veins, pseudoxanthoma elasticum, familial hyper-insulinemic hypoglycemia 1, Ullrich congenital muscular dystrophy, iminoglycinuria, Charge syndrome, Wilms Tumor, aniridia, genitourinary anomalies and mental retardation syndrome, tetralogy of Fallot, autosomal dominant spastic paraplegia 4, familial progressive scleroderma, Crest syndrome, autosomal dominant Emery-Dreifuss muscular dystrophy 2, aplasia of lacrimal and salivary glands, retinoblastoma, Dowling-Degos disease, primary pulmonary hypertension 1, Currarino syndrome, sacral agenesis syndrome, Prader-Willi syndrome, Greig cephalopolysyndactyly syndrome, juvenile polyposis/hereditary hemorrhagic telangiectasia syndrome, Piebald trait, limb-girdle muscular dystrophy type 1b, Bethlem myopathy, Cowden disease, Marfan syndrome, renal hypomagnesemia 2, microcephaly with or without chorioretinopathy, lymphedema or mental retardation tylosis with esophageal cancer, Kabuki syndrome 1, Jacobsen syndrome, diaphragmatic hernia, congenital Hashimoto thyroiditis, open angle glaucoma 1, Beckwith-Wiedemann syndrome, dopa-responsive dystonia, episodic kinesigenic dyskinesia 1, primary failure of tooth eruption, Darier-White disease, autosomal dominant cutis laxa 1, Cornelia De Lange syndrome 1, cleidocranial dysplasia, orofacial cleft 1, Van Der Woude syndrome 1, cherubism, cerebral cavernous malformations, familial hypertrophic cardiomyopathy 4, cardiofaciocutaneous syndrome, brachydactyly type D, basal cell nevus syndrome, achondroplasia, parietal foramina 2, Potocki-Shaffer syndrome, autosomal dominant congenital dyskeratosis 2, mental retardation with language impairment and autistic features, autosomal dominant anhidrotic ectodermal dysplasia with T-cell immunodeficiency, corticosteroid-binding globulin deficiency, choreoathetosis, hypothyroidism and neonatal respiratory distress, primary coenzyme Q10 deficiency 1, Duane-Radial Ray syndrome, familial hemiplegic migraine 2, mirror movements 1, Nager type acrofacial dysostosis 1, palmoplantar keratoderma punctate type Ia, and hypogonadotropic hypogonadism with or without anosmia 2.

Furthermore the invention relates to artificial transcription factors directed to the OPA1 promoter as described above for use of increasing OPA1 production, and for use in the treatment of diseases influenced by OPA1, in particular for use in the treatment of such eye diseases. Diseases modulated by OPA1 are autosomal dominant optic atrophy, autosomal dominant optic atrophy plus, as well as normal tension glaucoma.

Likewise the invention relates to a method of treating a disease influenced by OPA1 comprising administering a therapeutically effective amount of an artificial transcription factor of the invention to a patient in need thereof. In particular the invention relates to a method of treating neurodegeneration associated with normal tension glaucoma or dominant optic atrophy. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore the invention relates to artificial transcription factors directed to the the TGFbR1 promoter as described above for use of increasing or decreasing TGFbR1 production, and for use in the treatment of pathological processes influenced by TGFbR1, in particular of use in the treatment of such pathological processes in the eye. Pathological processes modulated by TGFbR1 are mal-adapted wound healing following eye surgery.

Likewise the invention relates to a method of treating a disease influenced by TGFBR1 comprising administering a therapeutically effective amount of an artificial transcription factor of the invention to a patient in need thereof. In particular the invention relates to a method of treating neurodegeneration associated with normal tension glaucoma or dominant optic atrophy. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

### Use of Artificial Transcription Factors in Plants

Furthermore the invention relates to the use of artificial transcription factors targeting plant promoters to improve gene product generation. Preferably, DNA encoding the artificial transcription factors is cloned into vectors for transformation of plant-colonizing microorganisms or plants. Alternatively, the artificial transcription factors are directly applied in suitable compositions for topical applications to plants.

### Use of Artificial Transcription Factors in Non-Human Animals

Furthermore the invention relates to the use of artificial transcription factors targeting non-human animal promoters, haploinsufficient, to enhance gene product generation. Preferably, the artificial transcription factors are directly applied in suitable compositions for topical applications to non-human animals in need thereof.

### Examples

### Cloning of DNA Plasmids

For all cloning steps, restriction endonucleases and T4 DNA ligase are purchased from New England Biolabs. Shrimp Alkaline Phosphatase (SAP) is from Promega. The high-fidelity Platinum Pfx DNA polymerase (Invitrogen) is applied in all standard PCR reactions. DNA fragments and plasmids are isolated according to the manufacturer's instructions using NucleoSpin Gel and PCR Clean-up kit, NucleoSpin Plasmid kit, or NucleoBond Xtra Midi Plus kit (Macherey-Nagel). Oligonucleotides are purchased from Sigma-Aldrich. All relevant DNA sequences of newly generated plasmids were verified by sequencing (Microsynth).

### Cloning of Hexameric Zinc Finger Protein Libraries for Yeast One Hybrid

Hexameric zinc finger protein libraries containing GNN and/or CNN and/or ANN binding zinc finger (ZF) modules are cloned according to Gonzalez B. et al, 2010, Nat Protoc 5, 791-810 with the following improvements. DNA sequences coding for GNN, CNN and ANN ZF modules were synthesized and inserted into pUC57 (GenScript) resulting in pAN1049 (SEQ ID NO: 264), pAN1073 (SEQ ID NO: 265) and pAN1670 (SEQ ID NO: 266), respectively. Stepwise assembly of zinc finger protein (ZFP) libraries is done in pBluescript SK (+) vector. To avoid insertion of multiple ZF modules during each individual cloning step leading to non-functional proteins, pBluescript (and its derived products containing 1ZFP, 2ZFPs, or 3ZFPs) and pAN1049, pAN1073 or pAN1670 are first incubated with one restriction enzyme and afterwards treated with SAP. Enzymes are removed using NucleoSpin Gel and PCR Clean-up kit before the second restriction endonuclease is added.

Cloning of pBluescript-1ZFPL is done by treating 5 μg pBluescript with XhoI, SAP and subsequently SpeI. Inserts are generated by incubating 10 μg pAN1049 (release of 16 different GNN ZF modules) or pAN1073 (release of 15 different CNN ZF modules) or pAN1670 (release of 15 different ANN ZF modules) with SpeI, SAP and subsequently XhoI. For generation of pBluescript-2ZFPL and pBluescript-3ZFPL, 7 μg pBluescript-1ZFPL or pBluescript-2ZFPL are cut with AgeI, dephosphorylated, and cut with SpeI. Inserts are obtained by applying SpeI, SAP, and subsequently XmaI to 10 μg pAN1049 or pAN1073 or pAN1670, respectively. Cloning of pBluescript-6ZFPL was done by treating 14 μg of pBluescript-3ZFPL with AgeI, SAP, and thereafter SpeI to obtain cut vectors. 3ZFPL inserts were released from 20 μg of pBluescript-3ZFPL by incubating with SpeI, SAP, and subsequently XmaI.

Ligation reactions for libraries containing one, two, and three ZFPs were set up in a 3:1 molar ratio of insert:vector using 200 ng cut vector, 400 U T4 DNA ligase in 20 μl total volume at RT (room temperature) overnight. Ligation reactions of hexameric zinc finger protein libraries included 2000 ng pBluescript-3ZFPL, 500 ng 3ZFPL insert, 4000 U T4 DNA ligase in 200 μl total volume, which were divided into ten times 20 μl and incubated separately at RT overnight. Portions of ligation reactions were transformed into Escherichia coli by several methods depending on the number of clones required for each library. For generation of pBluescript-1ZFPL and pBluescript-2ZFPL, 3 μl of ligation reaction were directly used for heat shock transformation of E. coli NEB 5-alpha. Plasmid DNA of ligation reactions of pBluescript-3ZFPL was purified using NucleoSpin Gel and PCR Clean-up kit and transformed into electrocompetent E. coli NEB 5-alpha (EasyjecT Plus electroporator from EquiBio or Multiporator from Eppendorf, 2.5 kV and 25 μF, 2 mm electroporation cuvettes from Bio-Rad). Ligation reactions of pBluescript-6ZFP libraries were applied to NucleoSpin Gel and PCR Clean-up kit and DNA was eluted in 15 μl of deionized water. About 60 ng of desalted DNA were mixed with 50 μl NEB 10-beta electrocompetent E. coli (New England Biolabs) and electroporation was performed as recommended by the manufacturer using EasyjecT Plus or Multiporator, 2.5 kV, 25 μF and 2 mm electroporation cuvettes. Multiple electroporations were performed for each library and cells were directly pooled afterwards to increase library size. After heat shock transformation or electroporation, SOC medium was applied to the bacteria and after 1 h of incubation at 37° C. and 250 rpm, 30 μl of SOC culture were used for serial dilutions and plating on LB plates containing ampicillin. The next day, total number of obtained library clones was determined. In addition, ten clones of each library were chosen to isolate plasmid DNA and to check incorporation of inserts by restriction enzyme digestion. At least three of these plasmids were sequenced to verify diversity of the library. The remaining SOC culture was transferred to 100 ml LB medium containing ampicillin and cultured overnight at 37° C. and 250 rpm. Those cells were used to prepare plasmid Midi DNA for each library.

For yeast one hybrid screens, hexameric zinc finger protein libraries are transferred to a compatible prey vector. For that purpose, the multiple cloning site of pGAD10 (Clontech) was modified by cutting the vector with XhoI/EcoRI and inserting annealed oligonucleotides OAN971 (TCGACAGGCCCAGGCGGCCCTCGAGGATATCATGATG ACTAGTGGCCAGGCCGGCCC, SEQ ID NO: 267) and OAN972 (AATTGGGCCGGC CTGGCCACTAGTCATCATGATATCCTCGAGGGCCGCCTGGGCCTG, SEQ ID NO: 268). The resulting vector pAN1025 (SEQ ID NO: 269) was cut and dephosphorylated, 6ZFP library inserts were released from pBluescript-6ZFPL by XhoI/SpeI. Ligation reactions and electroporations into NEB 10-beta electrocompetent E. coli were done as described above for pBluescript-6ZFP libraries.

For improved yeast one hybrid screening, hexameric zinc finger libraries are also transferred into an improved prey vector pAN1375 (SEQ ID NO: 270). This prey vector was constructed as follows: pRS315 (SEQ ID NO: 271) was cut ApaI/NarI and annealed OAN1143 (CGCCGCATGCATTCATGCAGGCC, SEQ ID NO: 272) and OAN1144 (TGCATGAATGCATGCGG, SEQ ID NO: 273) were inserted yielding pAN1373 (SEQ ID NO: 274). A SphI insert from pAN1025 was ligated into pAN1373 cut with SphI to obtain pAN1375.

For further improved yeast one hybrid screening, hexameric zinc finger libraries are also transferred into an improved prey vector pAN1920 (SEQ ID NO: 275).

For even further improved yeast one hybrid screening, hexameric zinc finger libraries are inserted into prey vector pAN1992 (SEQ ID NO: 276).

### Cloning of Bait Plasmids for Yeast One Hybrid Screening

For each bait plasmid, a 60 bp sequence containing a potential artificial transcription factor target site of 18 bp in the center is selected and a NcoI site is included for restriction analysis. Oligonucleotides are designed and annealed in such a way to produce 5′ HindIII and 3′ XhoI sites which allowed direct ligation into pAbAi (Clontech) cut with HindIII/XhoI. Digestion of the product with NcoI and sequencing are used to confirm assembly of the bait plasmid.

### Yeast Strain and Media

Saccharomyces cerevisiae Y1H Gold was purchased from Clontech, YPD medium and YPD agar from Carl Roth. Synthetic drop-out (SD) medium contained 20 g/l glucose, 6.8 g/l Na₂HPO₄.2H₂O, 9.7 g/l NaH₂PO₄.2H₂O (all from Carl Roth), 1.4 g/l yeast synthetic drop-out medium supplements, 6.7 g/l yeast nitrogen base, 0.1 g/l L-tryptophan, 0.1 g/l L-leucine, 0.05 g/l L-adenine, 0.05 g/l L-histidine, 0.05 g/l uracil (all from Sigma-Aldrich). SD-U medium contained all components except uracil, SD-L was prepared without L-leucine. SD agar plates did not contain sodium phosphate, but 16 g/l Bacto Agar (BD). Aureobasidin A (AbA) was purchased from Clontech.

### Preparation of Bait Yeast Strains

About 5 μg of each bait plasmid are linearized with BstBI in a total volume of 20 μl and half of the reaction mix is directly used for heat shock transformation of S. cerevisiae Y1H Gold. Yeast cells are used to inoculate 5 ml YPD medium the day before transformation and grown overnight on a roller at RT. One milliliter of this pre-culture is diluted 1:20 with fresh YPD medium and incubated at 30° C., 225 rpm for 2-3 h. For each transformation reaction 1 OD₆₀₀ cells are harvested by centrifugation, yeast cells are washed once with 1 ml sterile water and once with 1 ml TE/LiAc (10 mM Tris/HCl, pH 7.5, 1 mM EDTA, 100 mM lithium acetate). Finally, yeast cells are resuspended in 50 μl TE/LiAc and mixed with 50 μg single stranded DNA from salmon testes (Sigma-Aldrich), 10 μl of BstBI-linearized bait plasmid (see above), and 300 μl PEG/TE/LiAc (10 mM Tris/HCl, pH 7.5, 1 mM EDTA, 100 mM lithium acetate, 50% (w/v) PEG 3350). Cells and DNA are incubated on a roller for 20 min at RT, afterwards placed into a 42° C. water bath for 15 min. Finally, yeast cells are collected by centrifugation, resuspended in 100 μl sterile water and spread onto SD-U agar plates. After 3 days of incubation at 30° C. eight clones growing on SD-U from each transformation reaction are chosen to analyze their sensitivity towards aureobasidin A (AbA). Pre-cultures were grown overnight on a roller at RT. For each culture, OD₆₀₀ was measured and OD₆₀₀=0.3 was adjusted with sterile water. From this first dilution five additional 1:10 dilution steps were prepared with sterile water. For each clone 5 μl from each dilution step were spotted onto agar plates containing SD-U, SD-U 100 ng/ml AbA, SD-U 150 ng/ml AbA, and SD-U 200 ng/ml AbA. After incubation for 3 days at 30° C., three clones growing well on SD-U and being most sensitive to AbA are chosen for further analysis. Stable integration of bait plasmid into yeast genome is verified by Matchmaker Insert Check PCR Mix 1 (Clontech) according to the manufacturer's instructions. One of three clones is used for subsequent Y1H screen.

Transformation of Bait Yeast Strain with Hexameric Zinc Finger Protein Library

About 500 μl of yeast bait strain pre-culture are diluted into 1 I YPD medium and incubated at 30° C. and 225 rpm until OD₆₀₀=1.6-2.0 (circa 20 h). Cells are collected by centrifugation in a swing-out rotor (5 min, 1500×g, 4° C.). Preparation of electrocompetent cells is done according to Benatuil L. et al., 2010, Protein Eng Des Sel 23, 155-159. For each transformation reaction, 400 μl electrocompetent bait yeast cells are mixed with 1 μg prey plasmids encoding 6ZFP libraries and incubated on ice for 3 min. The cell-DNA suspension is transferred to a pre-chilled 2 mm electroporation cuvette. Multiple electroporation reactions (EasyjecT Plus electroporator or Multiporator, 2.5 kV and 25 μF) are performed until all yeast cell suspension has been transformed. After electroporation yeast cells are transferred to 100 ml of 1:1 mix of YPD:1 M sorbitol and incubated at 30° C. and 225 rpm for 60 min. Cells are collected by centrifugation and resuspended in 1-2 ml of SD-L medium. Aliquots of 200 μl are spread on 15 cm SD-L agar plates containing 1000-4000 ng/ml AbA. In addition, 50 μl of cell suspension are used to make 1/100 and 1/1000 dilutions and 50 μl of undiluted and diluted cells are plated on SD-L. All plates are incubated at 30° C. for 3 days. The total number of obtained clones is calculated from plates with diluted transformants. While SD-L plates with undiluted cells indicate growth of all transformants, AbA-containing SD-L plates only resulted in colony formation if the prey 6ZFP bound to its bait target site successfully.

### Verification of Positive Interactions and Recovery of 6ZFP-Encoding Prey Plasmids

For initial analysis, forty good-sized colonies are picked from SD-L plates containing the highest AbA concentration and yeast cells were restreaked twice on SD-L with 1000-4000 ng/ml AbA to obtain single colonies. For each clone, one colony is used to inoculate 5 ml SD-L medium and cells are grown at RT overnight. The next day, OD₆₀₀=0.3 is adjusted with sterile water, five additional 1/10 dilutions are prepared and 5 μl of each dilution step are spotted onto SD-L, SD-L 500 ng/ml AbA, 1000 ng/ml AbA, SD-L 1500 ng/ml AbA, SD-L 2000 ng/ml AbA, SD-L 2500 ng/ml AbA, SD-L 3000 ng/ml AbA, and SD-L 4000 ng/ml AbA plates. Clones are ranked according to their ability to grow on high AbA concentration. From best growing clones 5 ml of initial SD-L pre-culture are used to spin down cells and to resuspend them in 100 μl water or residual medium. After addition of 50 U lyticase (Sigma-Aldrich, L2524) cells are incubated for several hours at 37° C. and 300 rpm on a horizontal shaker. Generated spheroblasts are lysed by adding 10 μl 20% (w/v) SDS solution, mixed vigorously by vortexing for 1 min and frozen at −20° C. for at least 1 h. Afterwards, 250 μl Al buffer from NucleoSpin Plasmid kit and one spatula tip of glass beads (Sigma-Aldrich, G8772) are added and tubes are mixed vigorously by vortexing for 1 min. Plasmid isolation is further improved by adding 250 μl A2 buffer from NucleoSpin Plasmid kit and incubating for at least 15 min at RT before continuing with the standard NucleoSpin Plasmid kit protocol. After elution with 30 μl of elution buffer 5 μl of plasmid DNA are transformed into E. coli DH5 alpha by heat shock transformation. Two individual colonies are picked from ampicillin-containing LB plates, plasmids are isolated and library inserts are sequenced. Obtained results are analyzed for consensus sequences among the 6ZFPs for each target site.

### Cloning of Gene Promoters for Combined Secreted Luciferase and Alkaline Phosphatase Assay

DNA fragments containing promoter regions are cloned into pAN1485 (NEG-PG04, GeneCopeia) or pAN1486 (EF1a-PG04, GeneCopeia) resulting in reporter plasmids containing secreted Gaussia luciferase under the control of a haploinsufficient gene promoter and secreted embryonic alkaline phosphatase under the control of the constitutive CMV promoter allowing for normalization of luciferase to alkaline phosphatase signal.

### Cloning of a Reporter Plasmid for the Generation of Stable Luciferase/Secreted Alkaline Phosphatase Reporter Cell Lines for Testing Transducible Artificial Transcription Factor Activity

To generate a reporter construct containing Gaussia luciferase under the control of a hybrid CMV/artificial transcription factor target site promoter together with secreted alkaline phosphatase under control of the constitutive CMV promoter, 42 bp containing the artificial transcription factor binding site were cloned AflIII/SpeI into pAN1660 (SEQ ID NO: 277). These reporter constructs contain a FlpIn site for stable integration into FlpIn site containing cells such as HEK 293 FlpIn TRex (Invitrogen) cells.

### Cloning of Artificial Transcription Factors for Mammalian Transfection

DNA fragments encoding polydactyl zinc finger proteins either generated through Gensynthesis (GenScript) or selected by yeast one hybrid are cloned using standard procedures (AgeI/XhoI) into mammalian expression vectors for expression in mammalian cells as fusion proteins between the zinc finger array of interest, a SV40 NLS, a 3×myc epitope tag and a N-terminal KRAB domain (pAN1255—SEQ ID NO: 278), a C-terminal KRAB domain (pAN1258—SEQ ID NO: 279), a SID domain (pAN1257—SEQ ID NO: 280) or a VP64 activating domain (pAN1510—SEQ ID NO: 281).

Plasmids for the generation of stably transfected, tetracycline-inducible cells were generated as follows: DNA fragments encoding artificial transcriptions factors comprising polydactyl zinc finger domain, a regulatory domain (N-terminal KRAB, C-terminal KRAB, SID or VP64), SV40 NLS and a 3×myc epitope tag are cloned into pcDNA5/FRT/TO (Invitrogen) using EcoRV/NotI.

Plasmids for the generation of stably transfected, tetracycline-inducible cells were generated as follows: DNA fragments encoding artificial transcriptions factors comprising polydactyl zinc finger domain, a regulatory domain (N-terminal KRAB, C-terminal KRAB, SID or VP64), and a SV40 NLS are cloned into pAN2071 (SEQ ID NO: 282) EcoRV/AgeI. These artificial transcription factor expression plasmids can be integrated into the human genome into the AAVS1 locus by co-transfection with AAVS1 Left TALEN and AAVS1 Right TALEN (GeneCopoeia).

### Cell Culture and Transfections

HeLa cells are grown in Dulbecco's Modified Eagle's Medium (DMEM) supplemented with 4.5 g/l glucose, 10% heat-inactivated fetal bovine serum, 2 mM L-glutamine, and 1 mM sodium pyruvate (all from Sigma-Aldrich) in 5% CO₂ at 37° C. For luciferase reporter assay, 7000 HeLa cells/well are seeded into 96 well plates. Next day, co-transfections are performed using Effectene Transfection Reagent (Qiagen) according to the manufacturer's instructions. Plasmid midi preparations coding for artificial transcription factor and for luciferase are used in the ratio 3:1. Medium is replaced by 100 μl per well of fresh DMEM 6 h and 24 h after transfection.

### Generation and Maintenance of Flp-In™ T-Rex™ 293 Expression Cell Lines

Stable, tetracycline inducible Flp-In™ T-Rex™ 293 expression cell lines are generated by FlpIn Recombinase-mediated integration. Using Flp-In™ T-Rex™ Core Kit, the Flp-In™ T-Rex™ host cell line is generated by transfecting pFRT/lacZeo target site vector and pcDNA6/TR vector. For generation of inducible 293 expression cell lines, the pcDNA5/FRT/TO expression vector containing the gene of interest is integrated via Flp recombinase-mediated DNA recombination at the FRT site in the Flp-In™ T-Rex™ host cell line. Stable Flp-In™ T-Rex™ expression cell lines are maintained in selection medium containing (DMEM; 10% Tet-FBS; 2 mM glutamine; 15 μg/ml blasticidine and 100 μg/ml hygromycin). For induction of gene expression tetracycline is added to a final concentration of 1 μg/ml.

### Generation and Maintenance of Stably Artificial Transcription Factor Expressing Cell Lines Using TALENs

To generate cell lines stably expressing artificial transcription factors under the control of a tetracycline-inducible promoter, cells are co-transfected with a pAN2071-based expression construct containing the artificial transcription factor of interest and AAVS1 Left TALEN and AAVS1 Right TALEN (GeneCopoeia) plasmids using Effectene (Qiagen, transfection reagent) according to the manufacturer's recommendations. 8 hours post-transfection, growth medium was aspirated, cells were washed with PBS and fresh growth medium was added. 24 h post transfection cells were split at a ratio of 1:10 in growth medium containing Tet-approved FBS (tetracycline free FBS, Takara) without antibiotics. 48 h post-transfection, puromycin selection was started at cell-type specific concentration and cells were kept under selection pressure for 7-10 days. Colonies of stable cells were pooled and maintained in selection medium.

### Combined Luciferase/SEAP Promoter Activity Assay

HeLa cells are co-transfected with an artificial transcription factor expression construct and a plasmid carrying secreted Gaussia luciferase under the control of haploinsufficient promoter and secreted alkaline phosphatase under the control of the constitutive CMV promoter (Gaussia luciferase Glow Assay Kit, Pierce; SEAP Reporter Gene Assay chemiluminscent, Roche). Two days following transfection, cell culture supernatants were collected and luciferase activity and SEAP activity were measured using Secrete-Pair Dual Luminescence assay (GeneCopoeia) or SEAP reporter gene assay (Roche). Co-transfection of an expression plasmid for an inactive artificial transcription factor with all cysteine residues in the zinc finger domain exchanged to serine residues served as control. Luciferase activity was normalized to SEAP activity and expressed as percentage of control.

### Luciferase Reporter Assay for Assessing Artificial Transcription Factor Activity Following Protein Transduction

Stable HEK 293 FlpIn cells were prepared containing Gaussia luciferase under control of a hybrid CMV promoter containing the target site appropriate for the respective artificial transcription factor as well as SEAP under control of the constitutive CMV promoter. HEK 293 FlpIn cells were transfected with pAN1660, pAN2210 (SEQ ID NO: 283), pAN1705 (SEQ ID NO: 284), pAN2001 (SEQ ID NO: 285), pAN2122 (SEQ ID NO: 286), or pAN2100 (SEQ ID NO: 287), to generate cell lines for testing artificial transcription factors targeting ETRA (TS-74), ETRA (TS+50), FCER1A (TS-147), TLR4 (TS-222), TGFbR1 (TS-390), or AR (TS-236), respectively.

These cells were treated in OptiMem for 2 hours with the appropriate artificial transcription factor (1 μM) or with buffer, an unrelated or inactive artificial transcription factor as control. Following protein transduction, cells are harvested and reseeded into normal growth medium and luciferase as well as SEAP activity was measured after 24 hours according to manufacturer's recommendation (Gaussia Luciferase Glow Assay Kit, Thermo Scientific; SEAP Reporter Gene Assay Chemiluminescence, Roche). Luciferase values were normalized to SEAP activity and compared to control cells set to 100%.

### Determination of Gene Expression Levels by Quantitative RT-PCR

Total RNA is isolated from cells using the RNeasy Plus Mini Kit (Qiagen, Hilden, Germany) according to the manufacturer's instructions. Frozen cell pellets are resuspended in RLT Plus Lysis buffer containing 10 μl/ml β-mercaptoethanol. After homogenization using QIAshredder spin columns, total lysate is transferred to gDNA Eliminator spin columns to eliminate genomic DNA. One volume of 70% ethanol is added and total lysate is transferred to RNeasy spin columns. After several washing steps, RNA is eluted in a final volume of 30 μl RNase free water. RNA is stored at −80° C. until further use. Synthesis of cDNA is performed using the High Capacity cDNA Reverse Transcription Kit (Applied Biosystems, Branchburg, N.J., USA) according to the manufacturer's instructions. cDNA synthesis is carried out in 20 μl of total reaction volume containing 2 μl 10× Buffer, 0.8 μl 25×dNTP Mix, 2 μl 10×RT Random Primers, 1 μl Multiscribe Reverse Transcriptase and 4.2 μl H₂O. A final volume of 10 μl RNA is added and the reaction is performed under the following conditions: 10 minutes at 25° C., followed by 2 hours at 37° C. and a final step of 5 minutes at 85° C. Quantitative PCR is carried out in 20 μl of total reaction volume containing 1 μl 20×TaqMan Gene Expression Master Mix, 10.0 μl TaqMan® Universal PCR Master Mix (both Applied Biosystems, Branchburg, N.J., USA) and 8 μl H₂O. For each reaction 1 μl of cDNA is added. qPCR is performed using the ABI PRISM 7000 Sequence Detection System (Applied Biosystems, Branchburg, N.J., USA) under the following conditions: an initiation step for 2 minutes at 50° C. is followed by a first denaturation for 10 minutes at 95° C. and a further step consisting of 40 cycles of 15 seconds at 95° C. and 1 minute at 60° C.

### Cloning of Artificial Transcription Factors for Bacterial Expression

DNA fragments encoding artificial transcription factors are cloned using standard procedures with EcoRV/NotI into bacterial expression vector pAN983 (SEQ ID NO: 288) based on pET41a+ (Novagen) for expression in E. coli as His₆-tagged fusion proteins between the artificial transcription factor and the TAT protein transduction domain. For expression of cathepsin B sensitive artificial transcription factor containing a cathepsin B cleavage site of SEQ ID NO: 28, DNA fragments encoding artificial transcription factors are cloned using standard procedures (EcoRV/NotI) into bacterial expression vector pAN1688 (SEQ ID NO: 289).

Expression constructs for the bacterial production of cathepsin B-sensitive transducible artificial transcription factors in suitable E. coli host cells such as BL21(DE3) targeting ETRA, FcER1A, TLR4, AR, OPA1, or TGFbR1 are pAN1688, pAN1880 (SEQ ID NO: 290), pAN1966 (SEQ ID NO: 291), pAN2054 (SEQ ID NO: 292), pAN2056 (SEQ ID NO: 293), pAN2058 (SEQ ID NO: 294), pAN2060 (SEQ ID NO: 295), pAN2062 (SEQ ID NO: 296), pAN2064 (SEQ ID NO: 297), pAN2104 (SEQ ID NO: 298), pAN2112 (SEQ ID NO: 299), pAN2114 (SEQ ID NO: 300), pAN2116 (SEQ ID NO: 301), pAN2132 (SEQ ID NO: 302), pAN2134 (SEQ ID NO: 303), pAN2159 (SEQ ID NO: 304), pAN2160 (SEQ ID NO: 305), pAN2161 (SEQ ID NO: 306), pAN2286 (SEQ ID NO: 307), pAN2287 (SEQ ID NO: 308), pAN2288 (SEQ ID NO: 309), pAN2289 (SEQ ID NO: 310), pAN2290 (SEQ ID NO: 311), pAN2291 (SEQ ID NO: 312), pAN2292 (SEQ ID NO: 313), pAN2293 (SEQ ID NO: 314), pAN2323 (SEQ ID NO: 315), pAN2326 (SEQ ID NO: 316), pAN2328 (SEQ ID NO: 317), pAN2331 (SEQ ID NO: 318), and pAN2334 (SEQ ID NO: 319).

### Production of Artificial Transcription Factor Protein

E. coli BL21(DE3) transformed with expression plasmid for a given artificial transcription factor were grown in 1 I LB media supplemented with 100 μM ZnCl₂ until OD₆₀₀ between 0.8 and 1 was reached, and induced with 1 mM IPTG for two hours. Bacteria were harvested by centrifugation, bacterial lysate was prepared by sonication, and inclusion bodies were purified. To this end, inclusion bodies were collected by centrifugation (5000 g, 4° C., 15 minutes) and washed three times in 20 ml of binding buffer (50 mM HEPES, 500 mM NaCl, 10 mM imidazole; pH 7.5). Purified inclusion bodies were solubilized on ice for one hour in 30 ml of binding buffer A (50 mM HEPES, 500 mM NaCl, 10 mM imidazole, 6 M GuHCl; pH 7.5). Solubilized inclusion bodies were centrifuged for 40 minutes at 4° C. and 13'000 g and filtered through 0.45 μm PVDF filter. His-tagged artificial transcription factors were purified using His-Trap columns on an Äktaprime FPLC (GEHealthcare) using binding buffer A and elution buffer B (50 mM HEPES, 500 mM NaCl, 500 mM imidazole, 6 M GuHCl; pH 7.5). Fractions containing purified artificial transcription factor were pooled and dialyzed at 4° C. overnight against buffer S (50 mM Tris-HCl, 500 mM NaCl, 200 mM arginine, 100 μM ZnCl₂, 5 mM GSH, 0.5 mM GSSG, 50% glycerol; pH 7.5) in case the artificial transcription factor contained a SID domain, or against buffer K (50 mM Tris-HCl, 300 mM NaCl, 500 mM arginine, 100 μM ZnCl₂, 5 mM GSH, 0.5 mM GSSG, 50% glycerol; pH 8.5) for KRAB domain containing artificial transcription factors. Following dialysis, protein samples were centrifuged at 14'000 rpm for 30 minutes at 4° C. and sterile filtered using 0.22 μm Millex-GV filter tips (Millipore). For artificial transcription factors containing VP64 activation domain, the protein was produced from the soluble fraction (binding buffer: 50 mM NaPO₄ pH 7.5, 500 mM NaCl, 10 mM imidazole; elution buffer 50 mM HEPES pH 7.5, 500 mM NaCl, 500 mM imidazole) using His-Bond Ni-NTA resin (Novagen) according to manufactures recommendation. Protein was dialyzed against VP64-buffer (550 mM NaCl pH 7.4, 400 mM arginine, 100 μM ZnCl₂).

### Determination of DNA Binding Activity of Artificial Transcription Factors Using ELDIA (Enzyme-Linked DNA Interaction Assay)

BSA pre-blocked nickel coated plates (Pierce) are washed 3 times with wash buffer (25 mM Tris/HCl pH 7.5, 150 mM NaCl, 0.1% BSA, 0.05% Tween-20). Plates are coated with purified artificial transcription factor under saturating conditions (50 pmol/well) in storage buffer and incubated 1 h at RT with slight shake. After 3 washing steps, 1×10⁻¹² to 5×10⁻⁷ M of annealed, biotinylated oligos containing 60 bp promoter sequence are incubated in binding buffer (10 mM Tris/HCl pH 7.5, 60 mM KCl, 1 mM DTT, 2% glycerol, 5 mM MgCl₂ and 100 μM ZnCl₂) in the presence of unspecific competitor (0.1 mg/ml ssDNA from salmon sperm, Sigma) with the bound artificial transcription factor for 1 h at RT. After washing (5 times), wells are blocked with 3% BSA for 30 minutes at RT. Anti-streptavidin-HRP is added in binding buffer for 1 h at RT. After 5 washing steps, TMB substrate (Sigma) is added and incubated for 2 to 30 minutes at RT. Reaction is stopped by addition of TMB stop solution (Sigma) and sample extinction is read at 450 nm. Data analysis of ligand binding kinetics is done using Sigma Plot V8.1 according to Hill.

### Protein Transduction

Cells grown to about 80% confluency are treated with 0.01 to 1 μM artificial transcription factor or mock treated for 2 h to 120 h with optional addition of artificial transcription factor every 24 h in OptiMEM or growth media at 37° C. Optionally, 10-500 μM ZnCl₂ are added to the growth media. For immunofluorescence, cells are washed once in PBS, trypsinized and seeded onto glass cover slips for further examination.

### Immunofluorescence

Cells are fixed with 4% paraformaldehyde in PBS, treated with 0.15% Triton X-100 for 15 minutes, blocked with 10% BSA/PBS and incubated overnight with mouse anti-HA antibody (1:500, H9658, Sigma) or mouse anti-myc (1:500, M5546, Sigma). Samples are washed three times with PBS/1% BSA, and incubated with goat anti-mouse antibodies coupled to Alexa Fluor 546 (1:1000, Invitrogen) and counterstained using DAPI (1:1000 of 1 mg/ml for 3 minutes, Sigma). Samples are analyzed using fluorescence microscopy.

### Western Blotting

For measuring protein levels, cells are lysed using RIPA buffer (Pierce) and protein lysates are mixed with Laemmli sample buffer. Proteins are separated by SDS-PAGE according to their size and transferred using electroblotting to nitrocellulose membranes. Detection of proteins is performed using specific primary antibodies raised in mice or rabbits. Detection of primary antibodies is performed either by secondary antibodies coupled to horseradish peroxidase and luminescence-based detection (ECL plus, Pierce) or secondary antibodies coupled to DyLight700 or DyLight800 fluorescent detected and quantified using an infrared laser scanner.

### Measuring Mitochondrial Function

For flow cytometric analysis, treated cells are harvested with 10 mM EDTA/PBS. Mock treated cells are used as control. For measuring mitochondrial membrane potential, cells are resuspended in FACS buffer P (PBS, 5 mM EDTA, 0.5% (w/v) BSA, 1 μg/ml 4′,6-diamidino-2-phenylindole dihydrochloride (DAPI, Sigma), 10 nM tetramethylrhodamine ethylester (TMRE, Sigma) and incubated for 30 min at 37° C. prior to analysis. Treatment with 50 μM carbonyl cyanide 3-chlorophenylhydrazone (CCCP, Sigma) to dissipate mitochondrial membrane potential serves as control. For measurement of mitochondrial mass, cells are resuspended in FACS buffer M (PBS, 5 mM EDTA, 0.5% (w/v) BSA, 1 μg/ml DAPI and 100 nM MitoTracker green FM (Invitrogen) and incubated for 30 min at 37° C. prior to analysis. For mitochondrial ROS measurements, cells are resuspended in FACS buffer R (PBS, 5 mM EDTA, 0.5% BSA, 1 μg/ml DAPI and 5 μM MitoSOX (Invitrogen), incubated for 10 min at 37° C., washed with PBS, and resuspended in FACS buffer R2 (PBS, 5 mM EDTA, 0.5% (w/v) BSA). Flow cytometric analysis is performed on a CyAn_{ADP} (Dako) using FlowJo software (Tree Star Inc.).

### Measuring Cellular Apoptosis

Cells are fixed for 30 minutes at RT with 4% EM-grade paraformaldehyde (Pierce, 28908) in phosphate-buffered saline (PBS). Then, cells are permeabilized with 0.15% (v/v) Triton X-100 in PBS for 15 min at RT, followed by blocking with 10% (w/v) BSA in PBS for 1 hour at RT. Samples are incubated overnight at 4° C. with mouse anti-cytochrome c antibodies (BD Biosciences, 556432, 1:1000) diluted in blocking buffer. Cells are washed three times for 15 minutes with blocking buffer and then incubated for 1 hour at RT with Alexa Fluor 546-conjugated goat anti-mouse IgG antibodies (Invitrogen). Cytochrome c release as measure of apoptosis is analyzed by fluorescence microscopy by a blinded observer. Mock treated cells serve as control.

### Calcium Flux Measurements

Cells are seeded into 96-well Corning® CellBIND® plates and allowed to adhere in a humidified incubator (37° C.; 5% CO₂). The following day the cells are loaded using the Calcium 5 Assay Kit (Molecular Devices, CA, United States) as follows: For suspension cells the loading buffer is prepared as a two times solution in HBSS/20 mM HEPES (pH 7.4) and 100 μl/well are added to the wells containing 100 μl culture media. For adherent cells the loading buffer is prepared as a onetime solution in HBSS/20 mM HEPES (pH 7.4) and 100 μl/well are added to the wells directly after aspiration of culture media. When indicated, probenecid is added to the loading buffer to achieve a final in-well concentration of 2.5 mM. For dilution of ligands HBSS/20 mM HEPES (pH 7.4) is used. Calcium assays are carried out on a FlexStation® Instrument (Molecular Devices, CA, United States) according to the manufacturer's instructions. Data analysis is performed using SoftMax®Pro software.

Human Uterine Smooth Muscle Cells (hUtSMC) Lattice Contraction Assay 250 μl of sterile bovine collagen (3.1 mg/ml; #5005-B Nutacon) were mixed with 30 μl 10×PBS and 22.5 μl 0.1 N NaOH to reach a pH 7.4. 25000 hUtSMCs in 200 μl of SMC media 2 were added to the neutralized collagen, gently mixed, transferred to 24 well tissue culture plate and allowed to polymerize at 37° C., 5% CO₂ for 45 minutes. After polymerization, 500 μl of SMC growth media 2 were added. For treatment with artificial transcription factor, 1 μM ETRA+74VrepSNPS or an appropriate amount of buffer as control were added right after polymerization and again after 24 and 48 hours. 72 hours after polymerization, lattices were detached from the vessel wall by gently shaking or the help of a spatula and 100 nM of ET-1 or buffer control were added. Lattices were scanned and lattice area was determined by image analysis using ImageJ software.

### Human Coronary Contraction Assay

Human coronary arteries were dissected and cut into ring segments of approximately 2 mm length and placed individually into wells of a 96 well culture plate. Vessels were incubated in 250 μl of RPMI medium supplemented with penicillin (1000 IU/ml); streptomycin (100 μg/ml), amphotericin (0.25 μg/ml) and 1 μM ETRA+74VrepS or vehicle controls. Vessels were cultured for three days in an incubator at 37° C. in a humidified atmosphere of 5% CO₂ in air. Media was exchanged every 24 hours. One hour prior to media exchange, 3 nM endothelin were added to the vessels. Following incubation vessels were mounted in myograph baths (DMT) containing PSS (119.0 mM NaCl, 4.7 mM KCl, 1.2 mM MgSO₄, 24.9 mM NaHCO₃, 1.2 mM KH₂PO₄, 2.5 mM CaCl₂ and 11.1 mM glucose), aerated with 95% O₂ and 5% CO₂ and maintained at a temperature of 37° C. Tissues were exposed to potassium PSS (KPSS; 62.5 mM) three times, rinsed with PSS and allowed to return to baseline. Tissues were then exposed to U46619 (100 nM), followed by incubation with bradykinin (10 μM). Tissues were then rinsed, allowed to return to baseline and then exposed to the endothelin-1 in a cumulative concentration response curve (0, 1, 3, 10, 30, 100, 300 nM endothelin-1).

### Measuring Anaphylaxis in Humanized NSG Mice

Humanized NSG mice (2 animals/group—Jackson Laboratories) with an engraftment level of human CD45+ cells of at least 25% were treated with IgeR-147ArepS (30 mg/kg i.v.) or vehicle control 96 and 48 hours before induction of anaphylaxis. Mice were sensitized using anti-DNP IgE (3 μg i.v.) and treated with DNP-BSA (500 μg i.v.) or BSA (500 μg i.v.) as control to trigger anaphylaxis. Immediately after application of the anaphylactic trigger, body temperature was assessed by measuring rectal temperature every 5 minutes for 30 minutes and every 15 minutes for the next 90 minutes and every 30 minutes for the next two hours. In case of a drop in body temperature below 30° C., animals were euthanized.

1. An artificial transcription factor comprising a polydactyl zinc finger protein targeting specifically a gene promoter fused to an activatory or inhibitory protein domain, a nuclear localization sequence, a protein transduction domain, and an endosome-specific protease recognition site.

2. The artificial transcription factor according to claim 1, wherein the gene promoter is the promoter of a receptor gene.

3. The artificial transcription factor according to claim 1, wherein the gene promoter is the promoter of a nuclear receptor gene.

4. The artificial transcription factor according to claim 1, wherein the gene promoter is the promoter of a haploinsufficient gene.

5. The artificial transcription factor according to claim 1, wherein the endosome-specific protease recognition site is a cathepsin cleavage site.

6. The artificial transcription factor according to claim 5, wherein the endosome-specific protease recognition site is a cathepsin B cleavage site.

7. The artificial transcription factor according to claim 6, wherein the endosome-specific protease recognition site is a cathepsin B cleavage site of SEQ ID NO: 28.

8. The artificial transcription factor according to claim 1 comprising a zinc finger protein of a protein sequence selected from the group consisting of SEQ ID NO: 64 to 89, 144 to 147, 156 to 161, 174 to 177, 188, 191 to 193, 200 to 205, 218 to 220, and 227 to 244.

9. The artificial transcription factor according to claim 1 comprising a zinc finger protein of a protein sequence selected from the group consisting of SEQ ID NO: 64 to 89, 144 to 147, 156 to 161, 174 to 177, 188, 191 to 193, 200 to 205, 218 to 220, and 227 to 244, wherein up to three individual zinc finger modules are exchanged against other zinc finger modules with alternative binding characteristic and/or wherein up to twelve individual amino acids are exchanged.

10. The artificial transcription factor according to claim 1, wherein the activatory or inhibitory protein domain is selected from the group consisting of VP16, VP64, CJ7, p65-TA1, SAD, NF-1, AP-2, SP1-A, SP1-B, Oct-1, Oct-2, Oct2-5x, MTF-1, BTEB-2, LKLF, N-KRAB, C-KRAB, SID and ERD.

11. The artificial transcription factor according to claim 1, wherein the nuclear localization sequence is a cluster of basic amino acids containing a lysine residue followed by a lysine or arginine residue, followed by any amino acid, followed by a lysine or arginine residue, or the SV40 NLS of SEQ ID NO: 37.

12. The artificial transcription factor according to claim 1, wherein the protein transduction domain is selected from the group consisting of the HIV derived TAT peptide of SEQ ID NO: 20, mT02 of SEQ ID NO: 21, mT03 of SEQ ID NO: 22, R9 of SEQ ID NO: 23, and ANTP of SEQ ID NO: 24.

13. The artificial transcription factor according to claim 1 linked to a fusogenic peptide of SEQ ID NO: 25 to 27 through an endosomal protease-sensitive linker.

14. The artificial transcription factor according to claim 1 further comprising a polyethylene glycol residue.

15. The artificial transcription factor according to claim 1 for use in increasing or decreasing the expression from a gene promoter.

16. A pharmaceutical composition comprising an artificial transcription factor according to claim 1.

17. An E. coli host cell containing an expression construct of SEQ ID NO: 289 to 319 for use in the production of an artificial transcription factor of claim 1.

18. The artificial transcription factor according to claim 1 for use in treating a disease wherein modulation of gene expression is therapeutically beneficial.

19. A method of treating a disease wherein modulation of gene expression is therapeutically beneficial, comprising administering a therapeutically effective amount of an artificial transcription factor according to claim 1 to a patient in need thereof.
